(19) United States
(12) Patent Application Publication
(10) Pub. No.: US 2018/0133343 A1
(43) Pub. Date: May 17, 2018

(54) NANOPARTICLE CONJUGATES AND USES THEREOF

(71) Applicant: Massachusetts Institute of Technology, Cambridge, MA (US)

(72) Inventors: Darrell J. Irvine, Arlington, MA (US); Yu-Sang Sabrina Yang, Cambridge, MA (US)

(73) Assignee: Massachusetts Institute of Technology, Cambridge, MA (US)

(21) Appl. No.: 15/813,806

(22) Filed: Nov. 15, 2017

Related U.S. Application Data
(60) Provisional application No. 62/422,169, filed on Nov. 15, 2016.

(51) Int. Cl. A61K 47/69

(52) U.S. Cl. CPC A61K 47/6929; A61K 2039/505; A61K 47/6923

(57) ABSTRACT

The targeted delivery of therapeutic agents to specific cells remains a challenge in drug delivery. Provided herein are nanoparticle-targeting agent conjugates that can be used for the targeted delivery of therapeutic agents to certain cells and target tissues. The conjugates comprise nanoparticles (e.g., metal nanoparticles such as gold nanoparticles) with organic outer shells capable of adsorbing large numbers of therapeutic agents (e.g., small molecule drugs). The nanoparticles are covalently linked to targeting agents (e.g., proteins such as antibodies). The present invention also provides formulations comprising the nanoparticle-targeting agent conjugates, and kits comprising the same. In another aspect, the present invention provides methods of using the conjugates for the delivery of therapeutic agents to cells, and the treatment and/or prevention of diseases (e.g., autoimmune diseases, infectious diseases, proliferative diseases such as cancer). In another aspect, the present invention provides methods of preparing the nanoparticle-targeting agent conjugates described herein.

### RELATED APPLICATIONS

This application claims priority under 35 U.S.C. § 119(e) to U.S. Provisional Patent Application, U.S. Ser. No. 62/422,169, filed Nov. 15, 2016; the entire contents of which is incorporated herein by reference.

### GOVERNMENT SUPPORT

This invention was made with government support under contract number W911NF-13-D-0001 awarded by the Army Research Office. The government has certain rights in the invention.

### BACKGROUND OF THE INVENTION

The targeted delivery of therapeutic agents to specific cells remains a challenge in drug delivery. Small molecule drugs that modulate signaling pathways in lymphocytes represent a powerful strategy for enhancing anti-cancer immunity; however, many drugs have pleiotropic effects on other cells, leading to toxicities. One solution to this problem is to conjugate the drug to a targeting agent that will target specific cells. For example, antibody-drug conjugates (ADCs) are effective therapeutics that can maximize cytotoxin delivery to tumor cells while reducing off-target toxicities. Today there are two FDA-approved ADCs used in the clinic: Brentuximab vedotin and Trastuzumab emtansine. In addition, more than 30 ADCs are currently in clinical trials. Due to problems relating to antibody stability, however, current ADCs typically comprise one to four drug molecules per antibody. Given this limitation, drugs that are suitable for ADC development need to be highly potent.

Antibody-conjugated particles have been used as drug carriers to improve targeting efficiency. However, to date, most antibody-particle designs comprise a particle (50-200 nm) that is significantly bigger than an antibody (10-20 nm). This design motif creates a particular class of therapeutics with unideal biodistribution. In some instances, particles that are too large interfere with the targeting agent's (e.g., an antibody's) function and natural pharmacokinetics. Therefore, new drug delivery systems are needed which are capable of delivering therapeutic amounts of drugs with improved targeting efficiencies.

### SUMMARY OF THE INVENTION

Provided herein is a drug delivery system based on nanoparticle-targeting agent conjugates (“conjugates” herein) useful in the targeted delivery of therapeutic agents to cells. In one aspect, the present invention provides a conjugate comprising one or more nanoparticles covalently linked to a targeting agent, wherein each nanoparticle comprises an organic outer shell; and one or more therapeutic agents are associated with the organic outer shell of each nanoparticle. In certain embodiments, each nanoparticle is less than 10 nm in diameter. In certain embodiments, a conjugate provided herein comprises one or two nanoparticles that are about 3 nm in diameter. In particular embodiments, the nanoparticle is a gold nanoparticle that is less than 10 nm in diameter. In particular embodiments, the nanoparticle is a gold nanoparticle that is about 3 nm in diameter.

As described herein, one or more therapeutic agents are associated with the organic outer shell of each nanoparticle. The one or more therapeutic agents may be associated with the outer shell of the nanoparticle via covalent and/or non-covalent interactions. Nanoparticles comprising amphiphilic organic outer shells have dual properties: (1) the ability to adsorb hydrophobic drug molecules (i.e., via non-covalent interactions); and (2) the ability to disperse in cells following endocytic uptake by non-disruptively penetrating endosomal membranes. These amphiphilic nanoparticles can entrap tens to hundreds (or more) molecules in their hydrophobic ligand shells. In certain embodiments, the conjugates described herein comprise nanoparticles with amphiphilic outer shells, and have the capacity to load 50 or more (e.g., 50 to 200) molecules of a therapeutic agent per conjugate. In certain embodiments, the 50 or more molecules of a therapeutic agent are linked to the nanoparticle through covalent bonds (e.g., conjugates via covalent linking moieties).

Nanoparticles comprising amphiphilic outer shells can penetrate lipid bilayers without rupturing membranes, which can further enhance cytosolic delivery of the drug. In general, the nanoparticle-targeting agent conjugates provided herein provide a platform for directing and delivering a diverse range of therapeutic agents (e.g., small molecule drugs) to target cells for, e.g., direct modulation of cytosolic signaling pathways. Furthermore, conjugates of the present invention can be used to expand the diversity of therapeutic agents (e.g., small molecule drugs) that can be delivered with the help of targeting agents, especially those therapeutic agents that are hydrophobic and cannot be delivered efficiently otherwise.

In certain embodiments, the targeting agent covalently linked to the nanoparticle is an antibody. In such instances, the present invention can enable enhanced drug payload delivery (e.g., 100-fold) per antibody when compared with state of the art antibody-drug conjugates. Nanoparticle-antibody conjugates provided herein can enhance the therapeutic index of current antibody-drug conjugates, which have a limitation of carrying only 1-4 molecules per antibody. In particular embodiments, a nanoparticle (e.g., less than 10 nm in diameter) smaller than the antibody (e.g., more than 10 nm in length) is conjugated to the hinge region of the antibody. This construct preserves the antibody's natural pharmacokinetics and still has the capacity to deliver enhanced payloads to target cells. In certain embodiments, nanoparticle-antibody conjugates exhibit improved cellular targeting when compared with unmodified nanoparticles (e.g., nanoparticles without a targeting agent).

In addition to the conjugates themselves, the present invention also provides methods for preparing the conjugates described herein. In certain embodiments, a crosslinking reagent is used to conjugate a nanoparticle to a targeting agent, thereby covalently linking the nanoparticle to the targeting agent. Any bond forming reactions or techniques known in the art of bioconjugation can be used to prepare the conjugates described herein. For example, “click chemistry” reactions may be used.

The present invention also provides pharmaceutical compositions (i.e., formulations) comprising the conjugates described herein. The pharmaceutical composition may comprise a pharmaceutically acceptable excipient. The pharmaceutical compositions described herein may be useful for treating and/or preventing a disease (e.g., proliferative disease such as cancer, autoimmune disease, infectious disease) in a subject. The pharmaceutical compositions provided herein can comprise a therapeutically effective amount of a conjugate described herein. In certain embodiments, a pharmaceutical composition comprises a therapeutically effective amount of one or more therapeutic agents associated with the nanoparticle conjugates.

In another aspect, the present invention provides methods for delivering one or more therapeutic agents to a cell or target tissue, the methods comprising contacting the cell or target tissue with a conjugate described herein. The step of contacting can occur in vivo, in vitro, or ex vivo. In certain embodiments, the method comprises contacting a biological sample with a conjugate described herein. In certain embodiments, the one or more therapeutic agents are delivered to the cytosol of a cell. As described herein, the nanoparticle-targeting agent conjugates of the present invention can help confer improved cytosolic delivery of therapeutic agents.

In another aspect, the present invention provides methods for treating and/or preventing a disease in a subject, the methods comprising administering to the subject a conjugate described herein, or a pharmaceutical composition thereof. The conjugates provided herein can be used for the delivery of any therapeutic agent, and therefore are useful in the treatment and/or prevention of any disease or condition. In certain embodiments, the disease is a proliferative disease (e.g., cancer), an autoimmune disease, or an infectious disease. In certain embodiments, the method of treating and/or preventing a disease or condition in a subject comprises administering to the subject a therapeutically and/or prophylactically effective amount of a conjugate or pharmaceutical composition described herein.

Another aspect of the present invention relates to kits comprising a conjugate described herein, or a pharmaceutical composition thereof. The kits described herein may include a single dose or multiple doses of the conjugate or pharmaceutical composition described herein. The provided kits may be useful in a method of the invention (e.g., a method of treating and/or preventing a disease in a subject). A kit of the invention may further include instructions for using the kit (e.g., instructions for using the conjugate or pharmaceutical composition included in the kit).

The details of certain embodiments of the invention are set forth herein. Other features, objects, and advantages of the invention will be apparent from the Detailed Description, Figures, Examples, and Claims.

### Definitions

### General Definitions

The following definitions are general terms used throughout the present application.

“Antibody” (“antibodies” in the plural) refers to a class of proteins used by the immune system to identify and target particular agents, known as “antigens.” Generally, antibodies are immunoglobulin molecules capable of specific binding to targets through at least one antigen recognition site, located in the variable region of the immunoglobulin molecule. Antibodies typically have a Y shape, with each tip of the Y comprising a paratrope. A “paratrope” is a binding site on an antibody that binds to a particular site on an antigen, known as an “epitope.” Upon binding to an antigen, an antibody may alter the function of the antigen, impede or promote a biological process of the antigen, or recruit other agents (e.g., macrophages) to the antigen.

A typical antibody molecule comprises a heavy chain variable region (V_{H}) and a light chain variable region (V_{L}), which are usually involved in antigen binding. The V_{H} and V_{L} regions can be further subdivided into regions of hypervariability, also known as “complementarity determining regions” (“CDR”), interspersed with regions that are more conserved, which are known as “framework regions” (“FR”). Each V_{H} and V_{L} is typically composed of three CDRs and four FRs, arranged from amino-terminus to carboxy-terminus in the following order: FR1, CDR1, FR2, CDR2, FR3, CDR3, FR4. The extent of the framework region and CDRs can be precisely identified using methodology known in the art, for example, by the Kabat definition, the Chothia definition, the AbM definition, and/or the contact definition, all of which are well known in the art. See, e.g., Kabat, E. A., et al. (1991) Sequences of Proteins of Immunological Interest, Fifth Edition, U.S. Department of Health and Human Services, NIH Publication No. 91-3242, Chothia et al., (1989) Nature 342:877; Chothia, C. et al. (1987) J. Mol. Biol. 196:901-917, Al-lazikani et al (1997) J. Molec. Biol. 273:927-948; and Almagro, J. Mol. Recognit. 17:132-143 (2004). See also hgmp.mrc.ac.uk and bioinf.org.uk/abs).

An antibody useful in the present invention can be a full-length antibody, which contains two heavy chains and two light chains, each including a variable domain and a constant domain. Alternatively, the antibody can be an antigen-binding fragment of a full-length antibody. Examples of binding fragments encompassed within the term “antigen-binding fragment” of a full length antibody include, but are not limited to, (i) a Fab fragment, a monovalent fragment consisting of the V_{L}, V_{H}, C_{L} and C_{H}1 domains; (ii) a F(ab′)₂ fragment, a bivalent fragment including two Fab fragments linked by a disulfide bridge at the hinge region; (iii) a Fd fragment consisting of the V_{H} and C_{H}1 domains; (iv) a Fv fragment consisting of the V_{L} and V_{H} domains of a single arm of an antibody, (v) a dAb fragment (Ward et al., (1989) Nature 341:544-546), which consists of a V_{H} domain; and (vi) an isolated complementarity determining region (CDR) that retains functionality. Furthermore, although the two domains of the Fv fragment, V_{L} and V_{H}, are coded for by separate genes, they can be joined, using recombinant methods, by a synthetic linker that enables them to be made as a single protein chain in which the V_{L} and V_{H} regions pair to form monovalent molecules known as single chain Fv (scFv). See e.g., Bird et al. (1988) Science 242:423-426; and Huston et al. (1988) Proc. Natl. Acad. Sci. USA 85:5879-5883.

Further to the above, term “antibody” encompasses not only intact (i.e., full-length) polyclonal or monoclonal antibodies, but also antigen-binding fragments thereof (such as Fab, Fab′, F(ab′)₂, Fv), single chain (scFv), mutants thereof, fusion proteins comprising an antibody portion, humanized antibodies, chimeric antibodies, diabodies, linear antibodies, single chain antibodies, multispecific antibodies (e.g., bispecific antibodies), and any other modified configuration of the immunoglobulin molecule that comprises an antigen recognition site of the required specificity, including glycosylation variants of antibodies, amino acid sequence variants of antibodies, and covalently modified antibodies. In certain embodiments, the antibody is a single domain antibody (sdAb). An antibody includes an antibody of any class, such as IgD, IgE, IgG, IgA, or IgM (or sub-class thereof), and the antibody need not be of any particular class. Depending on the antibody amino acid sequence of the constant domain of its heavy chains, immunoglobulins can be assigned to different classes. There are five major classes of immunoglobulins: IgA, IgD, IgE, IgG, and IgM, and several of these may be further divided into subclasses (isotypes), e.g., IgG1, IgG2, IgG3, IgG4, IgA1 and IgA2. The heavy-chain constant domains that correspond to the different classes of immunoglobulins are called alpha, delta, epsilon, gamma, and mu, respectively. The subunit structures and three-dimensional configurations of different classes of immunoglobulins are well known.

The antibodies to be used in the systems described herein can be murine, rat, human, or any other origin (including chimeric or humanized antibodies). In some examples, the antibody comprises a modified constant region, such as a constant region that is immunologically inert, e.g., does not trigger complement mediated lysis, or does not stimulate antibody-dependent cell mediated cytotoxicity (ADCC). Any of the antibodies described herein can be either monoclonal or polyclonal. A “monoclonal antibody” refers to a homogenous antibody population and a “polyclonal antibody” refers to a heterogeneous antibody population. These two terms do not limit the source of an antibody or the manner in which it is made.

In certain embodiments, the antibody of a conjugate described herein is a humanized antibody. Humanized antibodies refer to forms of non-human (e.g., murine) antibodies that are specific chimeric immunoglobulins, immunoglobulin chains, or antigen-binding fragments thereof that contain minimal sequence derived from non-human immunoglobulin. For the most part, humanized antibodies are human immunoglobulins (recipient antibody) in which residues from a complementary determining region (CDR) of the recipient are replaced by residues from a CDR of a non-human species (donor antibody) such as mouse, rat, or rabbit having the desired specificity, affinity, and capacity. In some instances, Fv framework region (FR) residues of the human immunoglobulin are replaced by corresponding non-human residues. Furthermore, the humanized antibody may comprise residues that are found neither in the recipient antibody nor in the imported CDR or framework sequences, but are included to further refine and optimize antibody performance. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region or domain (Fc), typically that of a human immunoglobulin. Other forms of humanized antibodies have one or more CDRs (one, two, three, four, five, six) which are altered with respect to the original antibody, which are also termed one or more CDRs “derived from” one or more CDRs from the original antibody. Humanized antibodies may also involve affinity maturation.

In other embodiments, the antibody is a chimeric antibody, which can include a heavy constant region and a light constant region from a human antibody. Chimeric antibodies refer to antibodies having a variable region or part of variable region from a first species and a constant region from a second species. Typically, in these chimeric antibodies, the variable region of both light and heavy chains mimics the variable regions of antibodies derived from one species of mammals (e.g., a non-human mammal such as mouse, rabbit, and rat), while the constant portions are homologous to the sequences in antibodies derived from another mammal such as human. In some embodiments, amino acid modifications can be made in the variable region and/or the constant region.

An antibodies may specifically bind a target antigen. An antibody that “specifically binds” (used interchangeably herein) to a target or an epitope is a term well understood in the art, and methods to determine such specific binding are also well known in the art. A molecule is said to exhibit “specific binding” if it reacts or associates more frequently, more rapidly, with greater duration and/or with greater affinity with a particular target antigen than it does with alternative targets. An antibody “specifically binds” to a target antigen if it binds with greater affinity, avidity, more readily, and/or with greater duration than it binds to other substances. It is also understood by reading this definition that, for example, an antibody that specifically binds to a first target antigen may or may not specifically or preferentially bind to a second target antigen. As such, “specific binding” or “preferential binding” does not necessarily require (although it can include) exclusive binding. Generally, but not necessarily, reference to binding means preferential binding.

In certain embodiments, the targeting agent described herein is a monoclonal antibody. Examples of therapeutic monoclonal antibodies include, but are not limited to, Abagovomab, Abciximab, Adalimumab, Adecatumumab, Afelimomab, Afutuzumab, Alacizumab pegol, ALD, Alemtuzumab, Altumomab pentetate, Anatumomab mafenatox, Anrukinzumab, Anti-thymocyte globin, Apolizumab, Arcitumomab, Aselizumab, Atlizumab (tocilizumab), Atorolimumab, Bapineuzumab, Basiliximab, Bavituximab, Bectumomab, Belimumab, Benralizumab, Bertilimumab, Besilesomab, Bevacizumab, Biciromab, Bivatuzumab mertansine, Blinatumomab, Brentuximab vedotin, Briakinumab, Canakinumab, Cantuzumab mertansine, Capromab pendetide, Catumaxomab, Cedelizumab, Certolizumab pegol, Cetuximab, Citatuzumab bogatox, Cixutumumab, Clenoliximab, Clivatuzumab tetraxetan, Conatumumab, Dacetuzumab, Daclizumab, Daratumumab, Denosumab, Detumomab, Dorlimomab aritox, Dorlixizumab, Ecromeximab, Eculizumab, Edobacomab, Edrecolomab, Efalizumab, Efungumab, Elotuzumab, Elsilimomab, Enlimomab pegol, Epitumomab cituxetan, Epratuzumab, Erlizumab, Ertumaxomab, Etaracizumab, Exbivirumab, Fanolesomab, Faralimomab, Farletuzumab, Felvizumab, Fezakinumab, Figitumumab, Fontolizumab, Foravirumab, Fresolimumab, Galiximab, Gantenerumab, Gavilimomab, Gemtuzumab ozogamicin, GC1008, Girentuximab, Glembatumumab vedotin, Golimumab, Gomiliximab, Ibalizumab, Ibritumomab tiuxetan, Igovomab, Imciromab, Infliximab, Intetumumab, Inolimomab, Inotuzumab ozogamicin, Ipilimumab, Iratumumab, Keliximab, Labetuzumab, Lebrikizumab, Lemalesomab, Lerdelimumab, Lexatumumab, Libivirumab, Lintuzumab, Lorvotuzumab mertansine, Lucatumumab, Lumiliximab, Mapatumumab, Maslimomab, Matuzumab, Mepolizumab, Metelimumab, Milatuzumab, Minretumomab, Mitumomab, Morolimumab, Motavizumab, Muromonab-CD3, Nacolomab tafenatox, Naptumomab estafenatox, Natalizumab, Nebacumab, Necitumumab, Nerelimomab, Nimotuzumab, Nofetumomab merpentan, Ocrelizumab, Odulimomab, Ofatumumab, Olaratumab, Omalizumab, Oportuzumab monatox, Oregovomab, Otelixizumab, Pagibaximab, Palivizumab, Panitumumab, Panobacumab, Pascolizumab, Pemtumomab, Pertuzumab, Pexelizumab, Pintumomab, Priliximab, Pritumumab, Rafivirumab, Ramucirumab, Ranibizumab, Raxibacumab, Regavirumab Reslizumab, Rilotumumab, Rituximab, Robatumumab, Rontalizumab, Rovelizumab, Ruplizumab, Satumomab pendetide, Sevirumab, Sibrotuzumab, Sifalimumab, Siltuximab, Siplizumab, Solanezumab, Sonepcizumab, Sontuzumab, Stamulumab, Sulesomab, Tacatuzumab tetraxetan, Tadocizumab, Talizumab, Tanezumab, Taplitumomab paptox, Tefibazumab, Telimomab aritox, Tenatumomab, Teneliximab, Teplizumab, Ticilimumab (tremelimumab), Tigatuzumab, Tocilizumab (atlizumab), Toralizumab, Tositumomab, Trastuzumab, Tremelimumab, Tucotuzumab celmoleukin, Tuvirumab, Urtoxazumab, Ustekinumab, Vapaliximab, Vedolizumab, Veltuzumab, Vepalimomab, Visilizumab, Volociximab, Votumumab, Zalutumumab, Zanolimumab, Ziralimumab, and Zolimomab aritox. The targeting agent may be an antibody fragment, such as a fragment of any of the foregoing.

The “hinge region” of a Y-shaped antibody refers to the fork in the Y where the arms meet the stem. The hinge region is typically located between the C_{H}1 and C_{H2} domains of the antibody. The hinge region is flexible and allows the angle between the two arms of the antibody to vary.

“Antigen” refers to an agent which is targeted by and binds an antibody. In some instances, antigens trigger the immune system to produce antibodies against the antigens in what is known as an immune response. Common examples of antigens include foreign substances, such as bacteria and viruses; however; antigens need not be foreign substances. In certain embodiments, an antigen is expressed in a cell or on the surface of a cell. In certain embodiments, an antibody described herein is an antibody directed against a cluster of differentiation (CD) antigen (e.g., CD2, CD3, CD4, CD5, CD6, CD8, CD11, CD11a (LFA-1), CD15, CD18 (ITGB2), CD19, CD20 (MS4A1), CD22, CD23, CD25, CD27, CD28, CD30, CD33, CD37, CD38, CD40, CD41, CD44, CD49b (ITGA2), CD51, CD52, CD54 (ICAM-1), CD56, CD62L, CD70, CD74, CD79B, CD80, CD125, CD140a, CD142, CD147, CD152 (CTLA4), CD154, CD200, CD221, CD240D, CD248, CD257 (BAFF), CD274, CD276, CD279 (PDCD1)). Other examples of antigens which are common targets in therapeutics and drug delivery include, but are not limited to, glycoproteins (e.g., TPBG, EpCAM, CEA, gpA33, Mucins, TAG-72, CA-IX, CA-125 (MUC16), PSMA, endoglin, fibronectin, MUC1, mucin CanAg, rabies virus glycoprotein), glycolipids (e.g., gangliosides (e.g., GD2, GD3, GM2), myelin-associated glycoprotein, TAG-72, TN-C, TYRP1), carbohydrates (e.g., Lewis-Y²), folate binding proteins (e.g., folate receptor 1, folate receptor alpha), vascular targets (e.g., VEGF, VEGFR, αVβ3, α5β1, VAP-1, VEGF-A, VEGFR-1, VEGFR-2), growth factors (e.g., ErbB1/EGFR, ErbB2/HER2, ErbB3, c-MET, IGF1R, EphA3, TRAIL-R1, TRAIL-R2 (DR5), RANKL, EGFL7, GDF-8, HGF, HNGF, IGF-1, NGF, TGF-β, TGF-β1, TGF-β2), growth factor receptors (e.g., ERBB3, HER1, HER2/neu, HER3, HHGFR, IGF-1 receptor, PDGF-R α, PDGF-R β), stromal and extracellular matrix antigens (e.g., FAP, Tensacin), activin receptor (e.g., ACVR2B), activin receptor-like kinase (e.g., activing receptor-like kinase 2), angiopoetin (e.g., angiopoiein-2, angiopoetin-3), interferons (e.g., INF-α, INF-β, INF-γ), interleukeins (e.g., IL 17A, IL 17F, IL20, IL-12, IL-23, IL-1β, IL-17, IL-10, IL-22, IL-4, IL-5, IL-6, IL-6 receptor, IL-2, IL-23A, IL-31RA, IL-4, IL-6, IL-9, ILGF2), integrins (e.g., α₄β₇, α₅β₁, α₇β₇, α_{IIb}β₃, αᵥβ₃), complement component (e.g., C5, CFD), chemokines (e.g., CCL11, CCL2 (MCP-1)), chemokine receptors (e.g., CCR2, CCR4, CCR5), Notch receptors (e.g., Notch 1, NRP1), virulence factor (e.g., ClfA), colony stimulating factor (e.g., CSF2), colony stimulating factor receptors (e.g., CSF1R), delta-like ligands (e.g., DLL3, DLL4), Lipopolysaccharides (endotoxins), human leukocyte antigen (e.g., HLA-DR), heat shock proteins (e.g., Hsp90), SLAM proteins (e.g., SLAMF7), tissue factor pathway inhibitors, tumor necrosis factors (e.g., TNF-α), tumor necrosis factor receptors (TNFR superfamily member 4), microphage migration inhibitory factor, rhesus factor, neurite outgrowth inhibitor, alpha-fetoprotein, amyloid beta, carcinoembryonic antigen (CEA), neural apoptosis-regulated proteinase 1, Ch4D5, CLDN18.2, LOXL2, MSLN, NCA-90, PCSK9, sclerostin, syndecan 1, STEAP1, TSLP, TWEAK receptor, and tumor antigen CTAA16.88.

The term “particle” refers to a small object, fragment, or piece of a substance that may be a single element, inorganic material, organic material, or mixture thereof. Examples of particles include, but are not limited to, polymeric particles, single-emulsion particles, double-emulsion particles, coacervates, liposomes, microparticles, nanoparticles, macroscopic particles, pellets, crystals, aggregates, composites, pulverized, and cross-linked protein or polysaccharide particles. In certain embodiments, the particle is a metal particle. A metal particle may be made of a single metal, or a mixture of metals (e.g., alloy). In certain embodiments, the metal particle comprises a transition metal. Examples of metal particles include, but are not limited to, gold, silver, copper, platinum, palladium, ruthenium, rhenium, iron, and nickel particles.

The term “nanoparticle” refers to a particle having an average (e.g., mean) dimension (e.g., diameter) of between about 1 nanometer (nm) and about 1 micrometer (am) (e.g., between about 1 nm and about 300 nm, between about 1 nm and about 100 nm, between about 1 nm and about 30 nm, between about 1 nm and about 10 nm, or between about 1 nm and about 3 nm), inclusive. In certain embodiments, the nanoparticle is less than 10 nm in diameter. In certain embodiments, the nanoparticle is about 3 nm in diameter.

The term “small molecule” refers to molecules, whether naturally-occurring or artificially created (e.g., via chemical synthesis) that have a relatively low molecular weight. Typically, a small molecule is an organic compound (i.e., it contains carbon). The small molecule may contain multiple carbon-carbon bonds, stereocenters, and other functional groups (e.g., amines, hydroxyl, carbonyls, and heterocyclic rings, etc.). In certain embodiments, the molecular weight of a small molecule is not more than about 1,000 g/mol, not more than about 900 g/mol, not more than about 800 g/mol, not more than about 700 g/mol, not more than about 600 g/mol, not more than about 500 g/mol, not more than about 400 g/mol, not more than about 300 g/mol, not more than about 200 g/mol, or not more than about 100 g/mol. In certain embodiments, the molecular weight of a small molecule is at least about 100 g/mol, at least about 200 g/mol, at least about 300 g/mol, at least about 400 g/mol, at least about 500 g/mol, at least about 600 g/mol, at least about 700 g/mol, at least about 800 g/mol, or at least about 900 g/mol, or at least about 1,000 g/mol. Combinations of the above ranges (e.g., at least about 200 g/mol and not more than about 500 g/mol) are also possible. In certain embodiments, the small molecule is a therapeutically active agent such as a drug (e.g., a molecule approved by the U.S. Food and Drug Administration as provided in the Code of Federal Regulations (C.F.R.)). The small molecule may also be complexed with one or more metal atoms and/or metal ions. In this instance, the small molecule is also referred to as a “small organometallic molecule.” In certain embodiments, the small molecule is a drug. Preferably, though not necessarily, the drug is one that has already been deemed safe and effective for use in humans or animals by the appropriate governmental agency or regulatory body. For example, drugs approved for human use are listed by the FDA under 21 C.F.R. §§ 330.5, 331 through 361, and 440 through 460, incorporated herein by reference; drugs for veterinary use are listed by the FDA under 21 C.F.R. §§ 500 through 589, incorporated herein by reference. All listed drugs are considered acceptable for use in accordance with the present invention.

“Anti-cancer agents” are anti-proliferative agents and include chemotherapeutic agents. Exemplary chemotherapeutic agents include, but are not limited to, anti-estrogens (e.g. tamoxifen, raloxifene, and megestrol), LHRH agonists (e.g. goscrclin and leuprolide), anti-androgens (e.g. flutamide and bicalutamide), photodynamic therapies (e.g. vertoporfin (BPD-MA), phthalocyanine, photosensitizer Pc4, and demethoxy-hypocrellin A (2BA-2-DMHA)), nitrogen mustards (e.g. cyclophosphamide, ifosfamide, trofosfamide, chlorambucil, estramustine, and melphalan), nitrosoureas (e.g. carmustine (BCNU) and lomustine (CCNU)), alkylsulphonates (e.g. busulfan and treosulfan), triazenes (e.g. dacarbazine, temozolomide), platinum containing compounds (e.g. cisplatin, carboplatin, oxaliplatin), vinca alkaloids (e.g. vincristine, vinblastine, vindesine, and vinorelbine), taxoids (e.g. paclitaxel or a paclitaxel equivalent such as nanoparticle albumin-bound paclitaxel (ABRAXANE), docosahexaenoic acid bound-paclitaxel (DHA-paclitaxel, Taxoprexin), polyglutamate bound-paclitaxel (PG-paclitaxel, paclitaxel poliglumex, CT-2103, XYOTAX), the tumor-activated prodrug (TAP) ANG1005 (Angiopep-2 bound to three molecules of paclitaxel), paclitaxel-EC-1 (paclitaxel bound to the erbB2-recognizing peptide EC-1), and glucose-conjugated paclitaxel, e.g., 2′-paclitaxel methyl 2-glucopyranosyl succinate; docetaxel, taxol), epipodophyllins (e.g. etoposide, etoposide phosphate, teniposide, topotecan, 9-aminocamptothecin, camptoirinotecan, irinotecan, crisnatol, mytomycin C), anti-metabolites, DHFR inhibitors (e.g. methotrexate, dichloromethotrexate, trimetrexate, edatrexate), IMP dehydrogenase inhibitors (e.g. mycophenolic acid, tiazofurin, ribavirin, and EICAR), ribonuclotide reductase inhibitors (e.g. hydroxyurea and deferoxamine), uracil analogs (e.g. 5-fluorouracil (5-FU), floxuridine, doxifluridine, ratitrexed, tegafur-uracil, capecitabine), cytosine analogs (e.g. cytarabine (ara C), cytosine arabinoside, and fludarabine), purine analogs (e.g. mercaptopurine and Thioguanine), Vitamin D3 analogs (e.g. EB 1089, CB 1093, and KH 1060), isoprenylation inhibitors (e.g. lovastatin), dopaminergic neurotoxins (e.g. 1-methyl-4-phenylpyridinium ion), cell cycle inhibitors (e.g. staurosporine), actinomycin (e.g. actinomycin D, dactinomycin), bleomycin (e.g. bleomycin A2, bleomycin B2, peplomycin), anthracycline (e.g. daunorubicin, doxorubicin, pegylated liposomal doxorubicin, idarubicin, epirubicin, pirarubicin, zorubicin, mitoxantrone), MDR inhibitors (e.g. verapamil), Ca²⁺ ATPase inhibitors (e.g. thapsigargin), imatinib, thalidomide, lenalidomide, tyrosine kinase inhibitors (e.g., axitinib (AG013736), bosutinib (SKI-606), cediranib (RECENTIN™, AZD2171), dasatinib (SPRYCEL®, BMS-354825), erlotinib (TARCEVA®), gefitinib (IRESSA®), imatinib (Gleevec®, CGP57148B, STI-571), lapatinib (TYKERB®, TYVERB®), lestaurtinib (CEP-701), neratinib (HKI-272), nilotinib (TASIGNA®), semaxanib (semaxinib, SU5416), sunitinib (SUTENT®, SU11248), toceranib (PALLADIA®), vandetanib (ZACTIMA®, ZD6474), vatalanib (PTK787, PTK/ZK), trastuzumab (HERCEPTIN®), bevacizumab (AVASTIN®), rituximab (RITUXAN®), cetuximab (ERBITUX®), panitumumab (VECTIBIX®), ranibizumab (Lucentis®), nilotinib (TASIGNA®), sorafenib (NEXAVAR®), everolimus (AFINITOR®), alemtuzumab (CAMPATH®), gemtuzumab ozogamicin (MYLOTARG®), temsirolimus (TORISEL®), ENMD-2076, PCI-32765, AC220, dovitinib lactate (TKI258, CHIR-258), BIBW 2992 (TOVOK™), SGX523, PF-04217903, PF-02341066, PF-299804, BMS-777607, ABT-869, MP470, BIBF 1120 (VARGATEF®), AP24534, JNJ-26483327, MGCD265, DCC-2036, BMS-690154, CEP-11981, tivozanib (AV-951), OSI-930, MM-121, XL-184, XL-647, and/or XL228), proteasome inhibitors (e.g., bortezomib (VELCADE)), mTOR inhibitors (e.g., rapamycin, temsirolimus (CCI-779), everolimus (RAD-001), ridaforolimus, AP23573 (Ariad), AZD8055 (AstraZeneca), BEZ235 (Novartis), BGT226 (Norvartis), XL765 (Sanofi Aventis), PF-4691502 (Pfizer), GDC0980 (Genetech), SF1126 (Semafoe) and OSI-027 (OSI)), oblimersen, gemcitabine, carminomycin, leucovorin, pemetrexed, cyclophosphamide, dacarbazine, procarbizine, prednisolone, dexamethasone, campathecin, plicamycin, asparaginase, aminopterin, methopterin, porfiromycin, melphalan, leurosidine, leurosine, chlorambucil, trabectedin, procarbazine, discodermolide, carminomycin, aminopterin, and hexamethyl melamine.

As used herein, the term “salt” refers to any and all salts, and encompasses pharmaceutically acceptable salts.

The term “pharmaceutically acceptable salt” refers to those salts which are, within the scope of sound medical judgment, suitable for use in contact with the tissues of humans and lower animals without undue toxicity, irritation, allergic response, and the like, and are commensurate with a reasonable benefit/risk ratio. Pharmaceutically acceptable salts are well known in the art. For example, Berge et al. describe pharmaceutically acceptable salts in detail in J. Pharmaceutical Sciences, 1977, 66, 1-19, incorporated herein by reference. Pharmaceutically acceptable salts of the therapeutic agents used in this invention include those derived from suitable inorganic and organic acids and bases. Examples of pharmaceutically acceptable, nontoxic acid addition salts are salts of an amino group formed with inorganic acids, such as hydrochloric acid, hydrobromic acid, phosphoric acid, sulfuric acid, and perchloric acid or with organic acids, such as acetic acid, oxalic acid, maleic acid, tartaric acid, citric acid, succinic acid, or malonic acid or by using other methods known in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Salts derived from appropriate bases include alkali metal, alkaline earth metal, ammonium, and N+(C₁₋₄ alkyl)₄-salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate, and aryl sulfonate.

The terms “composition” and “formulation” are used interchangeably.

A “subject” to which administration is contemplated refers to a human (i.e., male or female of any age group, e.g., pediatric subject (e.g., infant, child, or adolescent) or adult subject (e.g., young adult, middle-aged adult, or senior adult)) or non-human animal. In certain embodiments, the non-human animal is a mammal (e.g., primate (e.g., cynomolgus monkey or rhesus monkey), commercially relevant mammal (e.g., cattle, pig, horse, sheep, goat, cat, or dog), or bird (e.g., commercially relevant bird, such as chicken, duck, goose, or turkey)). In certain embodiments, the non-human animal is a fish, reptile, or amphibian. The non-human animal may be a male or female at any stage of development. The non-human animal may be a transgenic animal or genetically engineered animal. The term “patient” refers to a human subject in need of treatment of a disease.

The term “biological sample” refers to any sample including tissue samples (such as tissue sections and needle biopsies of a tissue); cell samples (e.g., cytological smears (such as Pap or blood smears) or samples of cells obtained by microdissection); samples of whole organisms (such as samples of yeasts or bacteria); or cell fractions, fragments or organelles (such as obtained by lysing cells and separating the components thereof by centrifugation or otherwise). Other examples of biological samples include blood, serum, urine, semen, fecal matter, cerebrospinal fluid, interstitial fluid, mucous, tears, sweat, pus, biopsied tissue (e.g., obtained by a surgical biopsy or needle biopsy), nipple aspirates, milk, vaginal fluid, saliva, swabs (such as buccal swabs), or any material containing biomolecules that is derived from a first biological sample.

The term “target tissue” refers to any biological tissue of a subject (including a group of cells, a body part, or an organ) or a part thereof, including blood and/or lymph vessels, which is the object to which a conjugate, therapeutic agent, or composition thereof, is delivered. A target tissue may be an abnormal or unhealthy tissue, which may need to be treated. A target tissue may also be a normal or healthy tissue that is under a higher than normal risk of becoming abnormal or unhealthy, which may need to be prevented. A “non-target tissue” is any biological tissue of a subject (including a group of cells, a body part, or an organ) or a part thereof, including blood and/or lymph vessels, which is not a target tissue.

The term “administer,” “administering,” or “administration” refers to implanting, absorbing, ingesting, injecting, inhaling, or otherwise introducing a conjugate described herein, or a composition thereof, in or on a subject.

The terms “treatment,” “treat,” and “treating” refer to reversing, alleviating, delaying the onset of, or inhibiting the progress of a disease described herein. In some embodiments, treatment may be administered after one or more signs or symptoms of the disease have developed or have been observed. In other embodiments, treatment may be administered in the absence of signs or symptoms of the disease. For example, treatment may be administered to a susceptible subject prior to the onset of symptoms (e.g., in light of a history of symptoms and/or in light of exposure to a pathogen). Treatment may also be continued after symptoms have resolved, for example, to delay or prevent recurrence.

The terms “condition,” “disease,” and “disorder” are used interchangeably.

An “effective amount” of a conjugate described herein refers to an amount sufficient to elicit the desired biological response. An effective amount of a conjugate described herein may vary depending on such factors as the desired biological endpoint, the pharmacokinetics of the conjugate, the condition being treated, the mode of administration, and the age and health of the subject. In certain embodiments, an effective amount is a therapeutically effective amount. In certain embodiments, an effective amount is a prophylactic treatment. In certain embodiments, an effective amount is the amount of a conjugate described herein in a single dose. In certain embodiments, an effective amount is the combined amounts of a conjugate described herein in multiple doses.

The term “genetic disease” refers to a disease caused by one or more abnormalities in the genome of a subject, such as a disease that is present from birth of the subject. Genetic diseases may be heritable and may be passed down from the parents' genes. A genetic disease may also be caused by mutations or changes in the DNAs and/or RNAs of a subject. In such cases, the genetic disease will be heritable if it occurs in the germline. Exemplary genetic diseases include, but are not limited to, Aarskog-Scott syndrome, Aase syndrome, achondroplasia, acrodysostosis, addiction, adreno-leukodystrophy, albinism, ablepharon-macrostomia syndrome, alagille syndrome, alkaptonuria, alpha-1 antitrypsin deficiency, Alport's syndrome, Alzheimer's disease, asthma, autoimmune polyglandular syndrome, androgen insensitivity syndrome, Angelman syndrome, ataxia, ataxia telangiectasia, atherosclerosis, attention deficit hyperactivity disorder (ADHD), autism, baldness, Batten disease, Beckwith-Wiedemann syndrome, Best disease, bipolar disorder, brachydactyl), breast cancer, Burkitt lymphoma, chronic myeloid leukemia, Charcot-Marie-Tooth disease, Crohn's disease, cleft lip, Cockayne syndrome, Coffin Lowry syndrome, colon cancer, congenital adrenal hyperplasia, Cornelia de Lange syndrome, Costello syndrome, Cowden syndrome, craniofrontonasal dysplasia, Crigler-Najjar syndrome, Creutzfeldt-Jakob disease, cystic fibrosis, deafness, depression, diabetes, diastrophic dysplasia, DiGeorge syndrome, Down's syndrome, dyslexia, Duchenne muscular dystrophy, Dubowitz syndrome, ectodermal dysplasia Ellis-van Creveld syndrome, Ehlers-Danlos, epidermolysis bullosa, epilepsy, essential tremor, familial hypercholesterolemia, familial Mediterranean fever, fragile X syndrome, Friedreich's ataxia, Gaucher disease, glaucoma, glucose galactose malabsorption, glutaricaciduria, gyrate atrophy, Goldberg Shprintzen syndrome (velocardiofacial syndrome), Gorlin syndrome, Hailey-Hailey disease, hemihypertrophy, hemochromatosis, hemophilia, hereditary motor and sensory neuropathy (HMSN), hereditary non polyposis colorectal cancer (HNPCC), Huntington's disease, immunodeficiency with hyper-IgM, juvenile onset diabetes, Klinefelter's syndrome, Kabuki syndrome, Leigh's disease, long QT syndrome, lung cancer, malignant melanoma, manic depression, Marfan syndrome, Menkes syndrome, miscarriage, mucopolysaccharide disease, multiple endocrine neoplasia, multiple sclerosis, muscular dystrophy, myotrophic lateral sclerosis, myotonic dystrophy, neurofibromatosis, Niemann-Pick disease, Noonan syndrome, obesity, ovarian cancer, pancreatic cancer, Parkinson's disease, paroxysmal nocturnal hemoglobinuria, Pendred syndrome, peroneal muscular atrophy, phenylketonuria (PKU), polycystic kidney disease, Prader-Willi syndrome, primary biliary cirrhosis, prostate cancer, REAR syndrome, Refsum disease, retinitis pigmentosa, retinoblastoma, Rett syndrome, Sanfilippo syndrome, schizophrenia, severe combined immunodeficiency, sickle cell anemia, spina bifida, spinal muscular atrophy, spinocerebellar atrophy, sudden adult death syndrome, Tangier disease, Tay-Sachs disease, thrombocytopenia absent radius syndrome, Townes-Brocks syndrome, tuberous sclerosis, Turner syndrome, Usher syndrome, von Hippel-Lindau syndrome, Waardenburg syndrome, Weaver syndrome, Werner syndrome, Williams syndrome, Wilson's disease, xeroderma piginentosum, and Zellweger syndrome.

A “proliferative disease” refers to a disease that occurs due to abnormal growth or extension by the multiplication of cells (Walker, Cambridge Dictionary of Biology; Cambridge University Press: Cambridge, UK, 1990). A proliferative disease may be associated with: 1) the pathological proliferation of normally quiescent cells; 2) the pathological migration of cells from their normal location (e.g., metastasis of neoplastic cells); 3) the pathological expression of proteolytic enzymes such as the matrix metalloproteinases (e.g., collagenases, gelatinases, and elastases); or 4) the pathological angiogenesis as in proliferative retinopathy and tumor metastasis. Exemplary proliferative diseases include cancers (i.e., “malignant neoplasms”), benign neoplasms, diseases and conditions associated with angiogenesis, inflammatory diseases, and autoimmune diseases.

The term “angiogenesis” refers to the physiological process through which new blood vessels form from pre-existing vessels. Angiogenesis is distinct from vasculogenesis, which is the de novo formation of endothelial cells from mesoderm cell precursors. The first vessels in a developing embryo form through vasculogenesis, after which angiogenesis is responsible for most blood vessel growth during normal or abnormal development. Angiogenesis is a vital process in growth and development, as well as in wound healing and in the formation of granulation tissue. However, angiogenesis is also a fundamental step in the transition of tumors from a benign state to a malignant one, leading to the use of angiogenesis inhibitors in the treatment of cancer. Angiogenesis may be chemically stimulated by angiogenic proteins, such as growth factors (e.g., VEGF). “Pathological angiogenesis” refers to abnormal (e.g., excessive or insufficient) angiogenesis that amounts to and/or is associated with a disease.

The terms “neoplasm” and “tumor” are used herein interchangeably and refer to an abnormal mass of tissue wherein the growth of the mass surpasses and is not coordinated with the growth of a normal tissue. A neoplasm or tumor may be “benign” or “malignant,” depending on the following characteristics: degree of cellular differentiation (including morphology and functionality), rate of growth, local invasion, and metastasis. A “benign neoplasm” is generally well differentiated, has characteristically slower growth than a malignant neoplasm, and remains localized to the site of origin. In addition, a benign neoplasm does not have the capacity to infiltrate, invade, or metastasize to distant sites. Exemplary benign neoplasms include, but are not limited to, lipoma, chondroma, adenomas, acrochordon, senile angiomas, seborrheic keratoses, lentigos, and sebaceous hyperplasias. In some cases, certain “benign” tumors may later give rise to malignant neoplasms, which may result from additional genetic changes in a subpopulation of the tumor's neoplastic cells, and these tumors are referred to as “pre-malignant neoplasms.” An exemplary pre-malignant neoplasm is a teratoma. In contrast, a “malignant neoplasm” is generally poorly differentiated (anaplasia) and has characteristically rapid growth accompanied by progressive infiltration, invasion, and destruction of the surrounding tissue. Furthermore, a malignant neoplasm generally has the capacity to metastasize to distant sites. The term “metastasis,” “metastatic,” or “metastasize” refers to the spread or migration of cancerous cells from a primary or original tumor to another organ or tissue and is typically identifiable by the presence of a “secondary tumor” or “secondary cell mass” of the tissue type of the primary or original tumor and not of that of the organ or tissue in which the secondary (metastatic) tumor is located. For example, a prostate cancer that has migrated to bone is said to be metastasized prostate cancer and includes cancerous prostate cancer cells growing in bone tissue.

The term “cancer” refers to a class of diseases characterized by the development of abnormal cells that proliferate uncontrollably and have the ability to infiltrate and destroy normal body tissues. See, e.g., Stedman's Medical Dictionary, 25th ed.; Hensyl ed.; Williams & Wilkins: Philadelphia, 1990. Exemplary cancers include, but are not limited to, acoustic neuroma; adenocarcinoma; adrenal gland cancer; anal cancer; angiosarcoma (e.g., lymphangiosarcoma, lymphangioendotheliosarcoma, hemangiosarcoma); appendix cancer; benign monoclonal gammopathy; biliary cancer (e.g., cholangiocarcinoma); bladder cancer; breast cancer (e.g., adenocarcinoma of the breast, papillary carcinoma of the breast, mammary cancer, medullary carcinoma of the breast); brain cancer (e.g., meningioma, glioblastomas, glioma (e.g., astrocytoma, oligodendroglioma), medulloblastoma); bronchus cancer; carcinoid tumor; cervical cancer (e.g., cervical adenocarcinoma); choriocarcinoma; chordoma; craniopharyngioma; colorectal cancer (e.g., colon cancer, rectal cancer, colorectal adenocarcinoma); connective tissue cancer; epithelial carcinoma; ependymoma; endotheliosarcoma (e.g., Kaposi's sarcoma, multiple idiopathic hemorrhagic sarcoma); endometrial cancer (e.g., uterine cancer, uterine sarcoma); esophageal cancer (e.g., adenocarcinoma of the esophagus, Barrett's adenocarcinoma); Ewing's sarcoma; ocular cancer (e.g., intraocular melanoma, retinoblastoma); familiar hypereosinophilia; gall bladder cancer; gastric cancer (e.g., stomach adenocarcinoma); gastrointestinal stromal tumor (GIST); germ cell cancer; head and neck cancer (e.g., head and neck squamous cell carcinoma, oral cancer (e.g., oral squamous cell carcinoma), throat cancer (e.g., laryngeal cancer, pharyngeal cancer, nasopharyngeal cancer, oropharyngeal cancer)); hematopoietic cancers (e.g., leukemia such as acute lymphocytic leukemia (ALL) (e.g., B-cell ALL, T-cell ALL), acute myelocytic leukemia (AML) (e.g., B-cell AML, T-cell AML), chronic myelocytic leukemia (CML) (e.g., B-cell CML, T-cell CML), and chronic lymphocytic leukemia (CLL) (e.g., B-cell CLL, T-cell CLL)); lymphoma such as Hodgkin lymphoma (HL) (e.g., B-cell HL, T-cell HL) and non-Hodgkin lymphoma (NHL) (e.g., B-cell NHL such as diffuse large cell lymphoma (DLCL) (e.g., diffuse large B-cell lymphoma), follicular lymphoma, chronic lymphocytic leukemia/small lymphocytic lymphoma (CLL/SLL), mantle cell lymphoma (MCL), marginal zone B-cell lymphomas (e.g., mucosa-associated lymphoid tissue (MALT) lymphomas, nodal marginal zone B-cell lymphoma, splenic marginal zone B-cell lymphoma), primary mediastinal B-cell lymphoma, Burkitt lymphoma, lymphoplasmacytic lymphoma (i.e., Waldenstrim's macroglobulinemia), hairy cell leukemia (HCL), immunoblastic large cell lymphoma, precursor B-lymphoblastic lymphoma and primary central nervous system (CNS) lymphoma; and T-cell NHL such as precursor T-lymphoblastic lymphoma/leukemia, peripheral T-cell lymphoma (PTCL) (e.g., cutaneous T-cell lymphoma (CTCL) (e.g., mycosis fungoides, Sezary syndrome), angioimmunoblastic T-cell lymphoma, extranodal natural killer T-cell lymphoma, enteropathy type T-cell lymphoma, subcutaneous panniculitis-like T-cell lymphoma, and anaplastic large cell lymphoma); a mixture of one or more leukemia/lymphoma as described above; and multiple myeloma (MM)), heavy chain disease (e.g., alpha chain disease, gamma chain disease, mu chain disease); hemangioblastoma; hypopharynx cancer; inflammatory myofibroblastic tumors; immunocytic amyloidosis; kidney cancer (e.g., nephroblastoma a.k.a. Wilms' tumor, renal cell carcinoma); liver cancer (e.g., hepatocellular cancer (HCC), malignant hepatoma); lung cancer (e.g., bronchogenic carcinoma, small cell lung cancer (SCLC), non-small cell lung cancer (NSCLC), adenocarcinoma of the lung); leiomyosarcoma (LMS); mastocytosis (e.g., systemic mastocytosis); muscle cancer; myelodysplastic syndrome (MDS); mesothelioma; myeloproliferative disorder (MPD) (e.g., polycythemia vera (PV), essential thrombocytosis (ET), agnogenic myeloid metaplasia (AMM) a.k.a. myelofibrosis (MF), chronic idiopathic myelofibrosis, chronic myelocytic leukemia (CML), chronic neutrophilic leukemia (CNL), hypereosinophilic syndrome (HES)); neuroblastoma; neurofibroma (e.g., neurofibromatosis (NF) type 1 or type 2, schwannomatosis); neuroendocrine cancer (e.g., gastroenteropancreatic neuroendoctrine tumor (GEP-NET), carcinoid tumor); osteosarcoma (e.g., bone cancer); ovarian cancer (e.g., cystadenocarcinoma, ovarian embryonal carcinoma, ovarian adenocarcinoma); papillary adenocarcinoma; pancreatic cancer (e.g., pancreatic andenocarcinoma, intraductal papillary mucinous neoplasm (IPMN), Islet cell tumors); penile cancer (e.g., Paget's disease of the penis and scrotum); pinealoma; primitive neuroectodermal tumor (PNT); plasma cell neoplasia; paraneoplastic syndromes; intraepithelial neoplasms; prostate cancer (e.g., prostate adenocarcinoma); rectal cancer; rhabdomyosarcoma; salivary gland cancer; skin cancer (e.g., squamous cell carcinoma (SCC), keratoacanthoma (KA), melanoma, basal cell carcinoma (BCC)); small bowel cancer (e.g., appendix cancer); soft tissue sarcoma (e.g., malignant fibrous histiocytoma (MFH), liposarcoma, malignant peripheral nerve sheath tumor (MPNST), chondrosarcoma, fibrosarcoma, myxosarcoma); sebaceous gland carcinoma; small intestine cancer; sweat gland carcinoma; synovioma; testicular cancer (e.g., seminoma, testicular embryonal carcinoma); thyroid cancer (e.g., papillary carcinoma of the thyroid, papillary thyroid carcinoma (PTC), medullary thyroid cancer); urethral cancer; vaginal cancer; and vulvar cancer (e.g., Paget's disease of the vulva).

The term “inflammatory disease” refers to a disease caused by, resulting from, or resulting in inflammation. The term “inflammatory disease” may also refer to a dysregulated inflammatory reaction that causes an exaggerated response by macrophages, granulocytes, and/or T-lymphocytes leading to abnormal tissue damage and/or cell death. An inflammatory disease can be either an acute or chronic inflammatory condition and can result from infections or non-infectious causes. Inflammatory diseases include, without limitation, atherosclerosis, arteriosclerosis, autoimmune disorders, multiple sclerosis, systemic lupus erythematosus, polymyalgia rheumatica (PMR), gouty arthritis, degenerative arthritis, tendonitis, bursitis, psoriasis, cystic fibrosis, arthrosteitis, rheumatoid arthritis, inflammatory arthritis, Sjogren's syndrome, giant cell arteritis, progressive systemic sclerosis (scleroderma), ankylosing spondylitis, polymyositis, dermatomyositis, pemphigus, pemphigoid, diabetes (e.g., Type I), myasthenia gravis, Hashimoto's thyroiditis, Graves' disease, Goodpasture's disease, mixed connective tissue disease, sclerosing cholangitis, inflammatory bowel disease, Crohn's disease, ulcerative colitis, pernicious anemia, inflammatory dermatoses, usual interstitial pneumonitis (UIP), asbestosis, silicosis, bronchiectasis, berylliosis, talcosis, pneumoconiosis, sarcoidosis, desquamative interstitial pneumonia, lymphoid interstitial pneumonia, giant cell interstitial pneumonia, cellular interstitial pneumonia, extrinsic allergic alveolitis, Wegener's granulomatosis and related forms of angiitis (temporal arteritis and polyarteritis nodosa), inflammatory dermatoses, hepatitis, delayed-type hypersensitivity reactions (e.g., poison ivy dermatitis), pneumonia, respiratory tract inflammation, Adult Respiratory Distress Syndrome (ARDS), encephalitis, immediate hypersensitivity reactions, asthma, hayfever, allergies, acute anaphylaxis, rheumatic fever, glomerulonephritis, pyelonephritis, cellulitis, cystitis, chronic cholecystitis, ischemia (ischemic injury), reperfusion injury, allograft rejection, host-versus-graft rejection, appendicitis, arteritis, blepharitis, bronchiolitis, bronchitis, cervicitis, cholangitis, chorioamnionitis, conjunctivitis, dacryoadenitis, dermatomyositis, endocarditis, endometritis, enteritis, enterocolitis, epicondylitis, epididymitis, fasciitis, fibrositis, gastritis, gastroenteritis, gingivitis, ileitis, iritis, laryngitis, myelitis, myocarditis, nephritis, omphalitis, oophoritis, orchitis, osteitis, otitis, pancreatitis, parotitis, pericarditis, pharyngitis, pleuritis, phlebitis, pneumonitis, proctitis, prostatitis, rhinitis, salpingitis, sinusitis, stomatitis, synovitis, testitis, tonsillitis, urethritis, urocystitis, uveitis, vaginitis, vasculitis, vulvitis, vulvovaginitis, angitis, chronic bronchitis, osteomyelitis, optic neuritis, temporal arteritis, transverse myelitis, necrotizing fasciitis, and necrotizing enterocolitis. An ocular inflammatory disease includes, but is not limited to, post-surgical inflammation.

An “autoimmune disease” refers to a disease arising from an inappropriate immune response of the body of a subject against substances and tissues normally present in the body. In other words, the immune system mistakes some part of the body as a pathogen and attacks its own cells. This may be restricted to certain organs (e.g., in autoimmune thyroiditis) or involve a particular tissue in different places (e.g., Goodpasture's disease which may affect the basement membrane in both the lung and kidney). The treatment of autoimmune diseases is typically with immunosuppression, e.g., medications which decrease the immune response. Exemplary autoimmune diseases include, but are not limited to, glomerulonephritis, Goodpasture's syndrome, necrotizing vasculitis, lymphadenitis, peri-arteritis nodosa, systemic lupus erythematosis, rheumatoid arthritis, psoriatic arthritis, systemic lupus erythematosis, psoriasis, ulcerative colitis, systemic sclerosis, dermatomyositis/polymyositis, anti-phospholipid antibody syndrome, scleroderma, pemphigus vulgaris, ANCA-associated vasculitis (e.g., Wegener's granulomatosis, microscopic polyangiitis), uveitis, Sjogren's syndrome, Crohn's disease, Reiter's syndrome, ankylosing spondylitis, Lyme disease, Guillain-Barré syndrome, Hashimoto's thyroiditis, and cardiomyopathy.

The term “liver disease” or “hepatic disease” refers to damage to or a disease of the liver. Non-limiting examples of liver disease include intrahepatic cholestasis (e.g., alagille syndrome, biliary liver cirrhosis), fatty liver (e.g., alcoholic fatty liver, Reye's syndrome), hepatic vein thrombosis, hepatolenticular degeneration (i.e., Wilson's disease), hepatomegaly, liver abscess (e.g., amebic liver abscess), liver cirrhosis (e.g., alcoholic, biliary, and experimental liver cirrhosis), alcoholic liver diseases (e.g., fatty liver, hepatitis, cirrhosis), parasitic liver disease (e.g., hepatic echinococcosis, fascioliasis, amebic liver abscess), jaundice (e.g., hemolytic, hepatocellular, cholestatic jaundice), cholestasis, portal hypertension, liver enlargement, ascites, hepatitis (e.g., alcoholic hepatitis, animal hepatitis, chronic hepatitis (e.g., autoimmune, hepatitis B, hepatitis C, hepatitis D, drug induced chronic hepatitis), toxic hepatitis, viral human hepatitis (e.g., hepatitis A, hepatitis B, hepatitis C, hepatitis D, hepatitis E), granulomatous hepatitis, secondary biliary cirrhosis, hepatic encephalopathy, varices, primary biliary cirrhosis, primary sclerosing cholangitis, hepatocellular adenoma, hemangiomas, bile stones, liver failure (e.g., hepatic encephalopathy, acute liver failure), angiomyolipoma, calcified liver metastases, cystic liver metastases, fibrolamellar hepatocarcinoma, hepatic adenoma, hepatoma, hepatic cysts (e.g., Simple cysts, Polycystic liver disease, hepatobiliary cystadenoma, choledochal cyst), mesenchymal tumors (mesenchymal hamartoma, infantile hemangioendothelioma, hemangioma, peliosis hepatis, lipomas, inflammatory pseudotumor), epithelial tumors (e.g., bile duct hamartoma, bile duct adenoma), focal nodular hyperplasia, nodular regenerative hyperplasia, hepatoblastoma, hepatocellular carcinoma, cholangiocarcinoma, cystadenocarcinoma, tumors of blood vessels, angiosarcoma, Karposi's sarcoma, hemangioendothelioma, embryonal sarcoma, fibrosarcoma, leiomyosarcoma, rhabdomyosarcoma, carcinosarcoma, teratoma, carcinoid, squamous carcinoma, primary lymphoma, peliosis hepatis, erythrohepatic porphyria, hepatic porphyria (e.g., acute intermittent porphyria, porphyria cutanea tarda), and Zellweger syndrome.

The term “spleen disease” refers to a disease of the spleen. Example of spleen diseases include, but are not limited to, splenomegaly, spleen cancer, asplenia, spleen trauma, idiopathic purpura, Felty's syndrome, Hodgkin's disease, and immune-mediated destruction of the spleen.

The term “lung disease” or “pulmonary disease” refers to a disease of the lung. Examples of lung diseases include, but are not limited to, bronchiectasis, bronchitis, bronchopulmonary dysplasia, interstitial lung disease, occupational lung disease, emphysema, cystic fibrosis, acute respiratory distress syndrome (ARDS), severe acute respiratory syndrome (SARS), asthma (e.g., intermittent asthma, mild persistent asthma, moderate persistent asthma, severe persistent asthma), chronic bronchitis, chronic obstructive pulmonary disease (COPD), emphysema, interstitial lung disease, sarcoidosis, asbestosis, aspergilloma, aspergillosis, pneumonia (e.g., lobar pneumonia, multilobar pneumonia, bronchial pneumonia, interstitial pneumonia), pulmonary fibrosis, pulmonary tuberculosis, rheumatoid lung disease, pulmonary embolism, and lung cancer (e.g., non-small-cell lung carcinoma (e.g., adenocarcinoma, squamous-cell lung carcinoma, large-cell lung carcinoma), small-cell lung carcinoma).

A “hematological disease” includes a disease which affects a hematopoietic cell or tissue. Hematological diseases include diseases associated with aberrant hematological content and/or function. Examples of hematological diseases include diseases resulting from bone marrow irradiation or chemotherapy treatments for cancer, diseases such as pernicious anemia, hemorrhagic anemia, hemolytic anemia, aplastic anemia, sickle cell anemia, sideroblastic anemia, anemia associated with chronic infections such as malaria, trypanosomiasis, HTV, hepatitis virus or other viruses, myelophthisic anemias caused by marrow deficiencies, renal failure resulting from anemia, anemia, polycythemia, infectious mononucleosis (EVI), acute non-lymphocytic leukemia (ANLL), acute myeloid leukemia (AML), acute promyelocytic leukemia (APL), acute myelomonocytic leukemia (AMMoL), polycythemia vera, lymphoma, acute lymphocytic leukemia (ALL), chronic lymphocytic leukemia, Wilm's tumor, Ewing's sarcoma, retinoblastoma, hemophilia, disorders associated with an increased risk of thrombosis, herpes, thalassemia, antibody-mediated disorders such as transfusion reactions and erythroblastosis, mechanical trauma to red blood cells such as micro-angiopathic hemolytic anemias, thrombotic thrombocytopenic purpura and disseminated intravascular coagulation, infections by parasites such as Plasmodium, chemical injuries from, e.g., lead poisoning, and hypersplenism.

The term “neurological disease” refers to any disease of the nervous system, including diseases that involve the central nervous system (brain, brainstem and cerebellum), the peripheral nervous system (including cranial nerves), and the autonomic nervous system (parts of which are located in both central and peripheral nervous system). Neurodegenerative diseases refer to a type of neurological disease marked by the loss of nerve cells, including, but not limited to, Alzheimer's disease, Parkinson's disease, amyotrophic lateral sclerosis, tauopathies (including frontotemporal dementia), and Huntington's disease. Examples of neurological diseases include, but are not limited to, headache, stupor and coma, dementia, seizure, sleep disorders, trauma, infections, neoplasms, neuro-ophthalmology, movement disorders, demyelinating diseases, spinal cord disorders, and disorders of peripheral nerves, muscle and neuromuscular junctions. Addiction and mental illness, include, but are not limited to, bipolar disorder and schizophrenia, are also included in the definition of neurological diseases. Further examples of neurological diseases include acquired epileptiform aphasia; acute disseminated encephalomyelitis; adrenoleukodystrophy; agenesis of the corpus callosum; agnosia; Aicardi syndrome; Alexander disease; Alpers' disease; alternating hemiplegia; Alzheimer's disease; amyotrophic lateral sclerosis; anencephaly; Angelman syndrome; angiomatosis; anoxia; aphasia; apraxia; arachnoid cysts; arachnoiditis; Arnold-Chiari malformation; arteriovenous malformation; Asperger syndrome; ataxia telangiectasia; attention deficit hyperactivity disorder; autism; autonomic dysfunction; back pain; Batten disease; Behcet's disease; Bell's palsy; benign essential blepharospasm; benign focal; amyotrophy; benign intracranial hypertension; Binswanger's disease; blepharospasm; Bloch Sulzberger syndrome; brachial plexus injury; brain abscess; bbrain injury; brain tumors (including glioblastoma multiforme); spinal tumor; Brown-Sequard syndrome; Canavan disease; carpal tunnel syndrome (CTS); causalgia; central pain syndrome; central pontine myelinolysis; cephalic disorder; cerebral aneurysm; cerebral arteriosclerosis; cerebral atrophy; cerebral gigantism; cerebral palsy; Charcot-Marie-Tooth disease; chemotherapy-induced neuropathy and neuropathic pain; Chiari malformation; chorea; chronic inflammatory demyelinating polyneuropathy (CIDP); chronic pain; chronic regional pain syndrome; Coffin Lowry syndrome; coma, including persistent vegetative state; congenital facial diplegia; corticobasal degeneration; cranial arteritis; craniosynostosis; Creutzfeldt-Jakob disease; cumulative trauma disorders; Cushing's syndrome; cytomegalic inclusion body disease (CIBD); cytomegalovirus infection; dancing eyes-dancing feet syndrome; Dandy-Walker syndrome; Dawson disease; De Morsier's syndrome; Dejerine-Klumpke palsy; dementia; dermatomyositis; diabetic neuropathy; diffuse sclerosis; dysautonomia; dysgraphia; dyslexia; dystonias; early infantile epileptic encephalopathy; empty sella syndrome; encephalitis; encephaloceles; encephalotrigeminal angiomatosis; epilepsy; Erb's palsy; essential tremor; Fabry's disease; Fahr's syndrome; fainting; familial spastic paralysis; febrile seizures; Fisher syndrome; Friedreich's ataxia; frontotemporal dementia and other “tauopathies”; Gaucher's disease; Gerstmann's syndrome; giant cell arteritis; giant cell inclusion disease; globoid cell leukodystrophy; Guillain-Barre syndrome; HTLV-1 associated myelopathy; Hallervorden-Spatz disease; head injury; headache; hemifacial spasm; hereditary spastic paraplegia; heredopathia atactica polyneuritiformis; herpes zoster oticus; herpes zoster; Hirayama syndrome; HIV-associated dementia and neuropathy (see also neurological manifestations of AIDS); holoprosencephaly; Huntington's disease and other polyglutamine repeat diseases; hydranencephaly; hydrocephalus; hypercortisolism; hypoxia; immune-mediated encephalomyelitis; inclusion body myositis; incontinentia pigmenti; infantile; phytanic acid storage disease; Infantile Refsum disease; infantile spasms; inflammatory myopathy; intracranial cyst; intracranial hypertension; Joubert syndrome; Kearns-Sayre syndrome; Kennedy disease; Kinsbourne syndrome; Klippel Feil syndrome; Krabbe disease; Kugelberg-Welander disease; kuru; Lafora disease; Lambert-Eaton myasthenic syndrome; Landau-Kleffner syndrome; lateral medullary (Wallenberg) syndrome; learning disabilities; Leigh's disease; Lennox-Gastaut syndrome; Lesch-Nyhan syndrome; leukodystrophy; Lewy body dementia; lissencephaly; locked-in syndrome; Lou Gehrig's disease (aka motor neuron disease or amyotrophic lateral sclerosis); lumbar disc disease; lyme disease-neurological sequelae; Machado-Joseph disease; macrencephaly; megalencephaly; Melkersson-Rosenthal syndrome; Menieres disease; meningitis; Menkes disease; metachromatic leukodystrophy; microcephaly; migraine; Miller Fisher syndrome; mini-strokes; mitochondrial myopathies; Mobius syndrome; monomelic amyotrophy; motor neurone disease; moyamoya disease; mucopolysaccharidoses; multi-infarct dementia; multifocal motor neuropathy; multiple sclerosis and other demyelinating disorders; multiple system atrophy with postural hypotension; muscular dystrophy; myasthenia gravis; myelinoclastic diffuse sclerosis; myoclonic encephalopathy of infants; myoclonus; myopathy; myotonia congenital; narcolepsy; neurofibromatosis; neuroleptic malignant syndrome; neurological manifestations of AIDS; neurological sequelae of lupus; neuromyotonia; neuronal ceroid lipofuscinosis; neuronal migration disorders; Niemann-Pick disease; O'Sullivan-McLeod syndrome; occipital neuralgia; occult spinal dysraphism sequence; Ohtahara syndrome; olivopontocerebellar atrophy; opsoclonus myoclonus; optic neuritis; orthostatic hypotension; overuse syndrome; paresthesia; Parkinson's disease; paramyotonia congenita; paraneoplastic diseases; paroxysmal attacks; Parry Romberg syndrome; Pelizaeus-Merzbacher disease; periodic paralyses; peripheral neuropathy; painful neuropathy and neuropathic pain; persistent vegetative state; pervasive developmental disorders; photic sneeze reflex; phytanic acid storage disease; Pick's disease; pinched nerve; pituitary tumors; polymyositis; porencephaly; Post-Polio syndrome; postherpetic neuralgia (PHN); postinfectious encephalomyelitis; postural hypotension; Prader-Willi syndrome; primary lateral sclerosis; prion diseases; progressive; hemifacial atrophy; progressive multifocal leukoencephalopathy; progressive sclerosing poliodystrophy; progressive supranuclear palsy; pseudotumor cerebri; Ramsay-Hunt syndrome (Type I and Type II); Rasmussen's Encephalitis; reflex sympathetic dystrophy syndrome; Refsum disease; repetitive motion disorders; repetitive stress injuries; restless legs syndrome; retrovirus-associated myelopathy; Rett syndrome; Reye's syndrome; Saint Vitus Dance; Sandhoff disease; Schilder's disease; schizencephaly; septo-optic dysplasia; shaken baby syndrome; shingles; Shy-Drager syndrome; Sjogren's syndrome; sleep apnea; Soto's syndrome; spasticity; spina bifida; spinal cord injury; spinal cord tumors; spinal muscular atrophy; stiff-person syndrome; stroke; Sturge-Weber syndrome; subacute sclerosing panencephalitis; subarachnoid hemorrhage; subcortical arteriosclerotic encephalopathy; sydenham chorea; syncope; syringomyelia; tardive dyskinesia; Tay-Sachs disease; temporal arteritis; tethered spinal cord syndrome; Thomsen disease; thoracic outlet syndrome; tic douloureux; Todd's paralysis; Tourette syndrome; transient ischemic attack; transmissible spongiform encephalopathies; transverse myelitis; traumatic brain injury; tremor; trigeminal neuralgia; tropical spastic paraparesis; tuberous sclerosis; vascular dementia (multi-infarct dementia); vasculitis including temporal arteritis; Von Hippel-Lindau Disease (VHL); Wallenberg's syndrome; Werdnig-Hoffman disease; West syndrome; whiplash; Williams syndrome; Wilson's disease; and Zellweger syndrome.

A “painful condition” includes, but is not limited to, neuropathic pain (e.g., peripheral neuropathic pain), central pain, deafferentiation pain, chronic pain (e.g., chronic nociceptive pain, and other forms of chronic pain such as post-operative pain, e.g., pain arising after hip, knee, or other replacement surgery), pre-operative pain, stimulus of nociceptive receptors (nociceptive pain), acute pain (e.g., phantom and transient acute pain), noninflammatory pain, inflammatory pain, pain associated with cancer, wound pain, burn pain, postoperative pain, pain associated with medical procedures, pain resulting from pruritus, painful bladder syndrome, pain associated with premenstrual dysphoric disorder and/or premenstrual syndrome, pain associated with chronic fatigue syndrome, pain associated with pre-term labor, pain associated with withdrawal symptoms from drug addiction, joint pain, arthritic pain (e.g., pain associated with crystalline arthritis, osteoarthritis, psoriatic arthritis, gouty arthritis, reactive arthritis, rheumatoid arthritis or Reiter's arthritis), lumbosacral pain, musculo-skeletal pain, headache, migraine, muscle ache, lower back pain, neck pain, toothache, dental/maxillofacial pain, visceral pain and the like. One or more of the painful conditions contemplated herein can comprise mixtures of various types of pain provided above and herein (e.g. nociceptive pain, inflammatory pain, neuropathic pain, etc.). In some embodiments, a particular pain can dominate. In other embodiments, the painful condition comprises two or more types of pains without one dominating. A skilled clinician can determine the dosage to achieve a therapeutically effective amount for a particular subject based on the painful condition.

The term “psychiatric disorder” refers to a disease of the mind and includes diseases and disorders listed in the Diagnostic and Statistical Manual of Mental Disorders—Fourth Edition (DSM-IV), published by the American Psychiatric Association, Washington D. C. (1994). Psychiatric disorders include, but are not limited to, anxiety disorders (e.g., acute stress disorder agoraphobia, generalized anxiety disorder, obsessive-compulsive disorder, panic disorder, posttraumatic stress disorder, separation anxiety disorder, social phobia, and specific phobia), childhood disorders, (e.g., attention-deficit/hyperactivity disorder, conduct disorder, and oppositional defiant disorder), eating disorders (e.g., anorexia nervosa and bulimia nervosa), mood disorders (e.g., depression, bipolar disorder, cyclothymic disorder, dysthymic disorder, and major depressive disorder), personality disorders (e.g., antisocial personality disorder, avoidant personality disorder, borderline personality disorder, dependent personality disorder, histrionic personality disorder, narcissistic personality disorder, obsessive-compulsive personality disorder, paranoid personality disorder, schizoid personality disorder, and schizotypal personality disorder), psychotic disorders (e.g., brief psychotic disorder, delusional disorder, schizoaffective disorder, schizophreniform disorder, schizophrenia, and shared psychotic disorder), substance-related disorders (e.g., alcohol dependence, amphetamine dependence, cannabis dependence, cocaine dependence, hallucinogen dependence, inhalant dependence, nicotine dependence, opioid dependence, phencyclidine dependence, and sedative dependence), adjustment disorder, autism, delirium, dementia, multi-infarct dementia, learning and memory disorders (e.g., amnesia and age-related memory loss), and Tourette's disorder.

The term “metabolic disorder” refers to any disorder that involves an alteration in the normal metabolism of carbohydrates, lipids, proteins, nucleic acids, or a combination thereof. A metabolic disorder is associated with either a deficiency or excess in a metabolic pathway resulting in an imbalance in metabolism of nucleic acids, proteins, lipids, and/or carbohydrates. Factors affecting metabolism include, and are not limited to, the endocrine (hormonal) control system (e.g., the insulin pathway, the enteroendocrine hormones including GLP-1, PYY or the like), the neural control system (e.g., GLP-1 in the brain), or the like. Examples of metabolic disorders include, but are not limited to, diabetes (e.g., Type I diabetes, Type II diabetes, gestational diabetes), hyperglycemia, hyperinsulinemia, insulin resistance, and obesity.

A “diabetic condition” refers to diabetes and pre-diabetes. Diabetes refers to a group of metabolic diseases in which a person has high blood sugar, either because the body does not produce enough insulin, or because cells do not respond to the insulin that is produced. This high blood sugar produces the classical symptoms of polyuria (frequent urination), polydipsia (increased thirst) and polyphagia (increased hunger). There are several types of diabetes. Type I diabetes results from the body's failure to produce insulin, and presently requires the person to inject insulin or wear an insulin pump. Type II diabetes results from insulin resistance a condition in which cells fail to use insulin properly, sometimes combined with an absolute insulin deficiency. Gestational diabetes occurs when pregnant women without a previous diagnosis of diabetes develop a high blood glucose level. Other forms of diabetes include congenital diabetes, which is due to genetic defects of insulin secretion, cystic fibrosis-related diabetes, steroid diabetes induced by high doses of glucocorticoids, and several forms of monogenic diabetes, e.g., mature onset diabetes of the young (e.g., MODY 1, 2, 3, 4, 5, 6, 7, 8, 9, or 10). Pre-diabetes indicates a condition that occurs when a person's blood glucose levels are higher than normal but not high enough for a diagnosis of diabetes. All forms of diabetes increase the risk of long-term complications. These typically develop after many years, but may be the first symptom in those who have otherwise not received a diagnosis before that time. The major long-term complications relate to damage to blood vessels. Diabetes doubles the risk of cardiovascular disease and macrovascular diseases such as ischemic heart disease (angina, myocardial infarction), stroke, and peripheral vascular disease. Diabetes also causes microvascular complications, e.g., damage to the small blood vessels. Diabetic retinopathy, which affects blood vessel formation in the retina of the eye, can lead to visual symptoms, reduced vision, and potentially blindness. Diabetic nephropathy, the impact of diabetes on the kidneys, can lead to scarring changes in the kidney tissue, loss of small or progressively larger amounts of protein in the urine, and eventually chronic kidney disease requiring dialysis. Diabetic neuropathy is the impact of diabetes on the nervous system, most commonly causing numbness, tingling and pain in the feet and also increasing the risk of skin damage due to altered sensation. Together with vascular disease in the legs, neuropathy contributes to the risk of diabetes-related foot problems, e.g., diabetic foot ulcers, that can be difficult to treat and occasionally require amputation.

“Infectious disease” refers to a disease which results from an infection. An infection is a condition caused by the invasion of an organism by a foreign agent (i.e., infectious agent). Infectious agents include, but are not limited to, bacteria, fungi, viruses, viroids, nematodes (e.g., parasites such as roundworms and pinworms), anthropods (e.g., mites, fleas, lice, ticks), and macroparasites (e.g., tapeworms). Common infectious diseases include bacterial and viral infections.

### Chemical Definitions

“Hydrophobic” or “lipophilic” refers to the ability of a compound to dissolve—or the ability of a moiety of a compound to assist the compound in dissolving—in fats, oils, lipids, and/or non-polar solvents. Hydrophobic compounds or moieties typically have a relatively high octanol/water partition coefficient. In certain embodiments, hydrophobic moieties are substituted or unsubstituted, branched or unbranched, aliphatic groups. In certain embodiments, hydrophobic moieties are unsubstituted, branched or unbranched, aliphatic groups. The term “aliphatic” refers to alkyl, alkenyl, alkynyl, and carbocyclyl groups, and combinations thereof. Hydrophobic moieties include, but are not limited to, unsubstituted, branched or unbranched aliphatic groups having 1 to 50 carbon atoms. In certain embodiments, a hydrophobic moiety is an unsubstituted, branched or unbranched alkyl group having 1 to 50 carbon atoms. In certain embodiments, the hydrophobic moiety is unsubstituted, branched or unbranched C₁₋₂₄ alkyl. In certain embodiments, the hydrophobic moiety is unsubstituted branched or unbranched C₆₋₂₄ alkyl. In certain embodiments, the hydrophobic moiety is unsubstituted branched or unbranched C₆₋₁₀ alkyl.

“Hydrophilic” refers to the ability of a compound or moiety to mix with or dissolve in water. It may also refer to the ability of a group on a compound to assist the compound in mixing or dissolving with water. Typically, hydrophilic compounds or moieties typically have a relatively low octanol/water partition coefficient. Hydrophilic moieties comprise one or more hydrogen bond donor groups and/or one or more hydrogen-bond acceptor groups. Hydrogen bond donor and acceptor groups are typically heteroatom-containing groups (e.g., hydroxyl, amino, thio, sulfonate, sulfinate, carbonyl, phosphate, oxo groups). In certain embodiments, hydrophilic moieties are substituted, branched or unbranched, aliphatic groups. In certain embodiments, hydrophilic moieties are substituted, branched or unbranched alkyl groups. In certain embodiments, the hydrophilic moiety is substituted, branched or unbranched alkyl. In certain embodiments, the hydrophilic moiety is substituted, branched or unbranched C₁₋₂₄ alkyl. In certain embodiments, the hydrophilic moiety is substituted, branched or unbranched C₆₋₂₄ alkyl. In certain embodiments, the hydrophobic moiety is substituted, branched or unbranched C₆₋₁₀ alkyl. Exemplary substituents (e.g., hydrogen bond donors and/or acceptors) include, but are not limited to, —CN, —NO₂, —N₃, —SO₂H, —SO₃H, —OH, —OR, —ON(R)₂, —N(R)₂, —N(R)₃⁺X⁻, —N(OR)R, —SH, —SR, —SSR, —C(═O)R, —CO₂H, —CHO, —C(OR)₂, —CO₂R, —OC(═O)R, —OCO₂R, —C(═O)N(R)₂, —OC(═O)N(R)₂, —NRC(═O)R, —NRCO₂R, —NRC(═O)N(R)₂, —C(═NR)R, —C(═NR)OR, —OC(═NR)R, —OC(═NR)OR, —C(═NR)N(R)₂, —OC(═NR)N(R)₂, —NRC(═NR)N(R)₂, —C(═O)NRSO₂R, —NRSO₂R, —SO₂N(R)₂, —SO₂R, —SO₂OR, —OSO₂R, —S(═O)R, —OS(═O)R, —Si(R)₃, —OSi(R)₃—C(═S)N(R)₂, —C(═O)SR, —C(═S)SR, —SC(═S)SR, —SC(═O)SR, —OC(═O)SR, —SC(═O)OR, —SC(═O)R, —P(═O)(R)₂, —P(═O)(OR)₂, —OP(═O)(R)₂, —OP(═O)(OR)₂, —P(═O)(N(R)₂)₂, —OP(═O)(N(R)₂)₂, —NRP(═O)(R)₂, —NRP(═O)(OR)₂, —NRP(═O)(N(R)₂)₂, —P(R)₂, —P(OR)₂, —P(R)₃⁺X⁻, —P(OR)₃⁺X⁻, —P(R)₄, —P(OR)₄, —OP(R)₂, —OP(R)₃⁺X⁻, —OP(OR)₂, —OP(OR)₃⁺X⁻, —OP(R)₄, —OP(OR)₄, —B(R)₂, —B(OR)₂, —BR(OR); or two geminal hydrogens on a carbon atom are replaced with the group ═O, ═S, ═NN(R)₂, ═NNRC(═O)R, ═NNRC(═O)OR, ═NNRS(═O)₂R, ═NR, or ═NOR; wherein each instance of R is independently hydrogen, alkyl, alkenyl, alkynyl, aryl, heteroaryl, carbocyclyl, heterocyclyl, acyl, or a protecting group; and wherein X⁻ is a counterion.

“Click chemistry” refers to a chemical approach to conjugation introduced by Sharpless in 2001 and describes chemistry tailored to generate substances quickly and reliably by joining units together. See, e.g., Kolb, Finn and Sharpless Angewandte Chemie International Edition 2001 40, 2004-2021; Evans, Australian Journal of Chemistry 2007 60, 384-395). Exemplary coupling reactions (some of which may be classified as “click chemistry”) include, but are not limited to, formation of esters, thioesters, amides (e.g., such as peptide coupling) from activated acids or acyl halides; nucleophilic displacement reactions (e.g., such as nucleophilic displacement of a halide or ring opening of strained ring systems); azide-alkyne Huisgen cycloaddition; thiol-yne addition; imine formation; Michael additions (e.g., maleimide addition reactions); and Diels-Alder reactions (e.g., tetrazine [4+2] cycloaddition). Examples of click chemistry reactions can be found in, e.g., Kolb, H. C.; Finn, M. G. and Sharpless, K. B. Angew. Chem. Int. Ed. 2001, 40, 2004-2021. Kolb, H. C. and Sharless, K. B. Drug Disc. Today, 2003, 8, 112-1137; Rostovtsev, V. V.; Green L. G.; Fokin, V. V. and Shrapless, K. B. Angew. Chem. Int. Ed. 2002, 41, 2596-2599; Tomoe, C. W.; Christensen, C. and Meldal, M. J. Org. Chem. 2002, 67, 3057-3064. Wang, Q. et al. J. Am. Chem. Soc. 2003, 125, 3192-3193; Lee, L. V. et al. J. Am. Chem. Soc. 2003 125, 9588-9589; Lewis, W. G. et al. Angew. Chem. Int. Ed. 2002, 41, 1053-41057; Manetsch, R. et al., J. Am. Chem. Soc. 2004, 126, 12809-12818; Mocharla, V. P. et al. Angew. Chem., Int. Ed. 2005, 44, 116-120.

These and other exemplary substituents are described in more detail in the Detailed Description, Examples, and Claims. The invention is not intended to be limited in any manner by the above exemplary listing of substituents.

### BRIEF DESCRIPTION OF THE DRAWINGS

The accompanying drawings, which constitute a part of this Application, illustrate several embodiments of the invention and together with the description, serve to explain the principles of the invention.

FIGS. 1A-1D show design, quantification, and functionality test of nanoparticle-antibody conjugates (also “antibody drug nanocarriers” (ADNCs)). Schematic illustration of anti-CD8 whole IgG antibody or single domain V_{H}H antibody conjugation to amphiphilic nanoparticles. FIG. 1A is an illustration of amphiphilic nanoparticle (amph-NP) surface modification chemistry. A 3-step chemical modification process is shown: ligand exchange to attach NH₂ functional group to amph-NPs, followed by maleimide functionalization using a crosslinking reagent, and finally conjugation to the reactive moieties on an antibody. For example, gold amph-NPs are modified with 11-amino-1-undecanethiol hydrochloride. Then, free amines on the outer shell of the nanoparticles are capped by sulfo-MBS and coupled to hinge region thiols of mildly reduced antibodies. FIG. 1B and FIG. 1C show SDSPAGE quantification of mass ratio of antibody to gold nanoparticles (220 μg Antibody per mg nanoparticle). FIG. 1B shows an SDS-PAGE image of anti-CD8 IgG antibody-conjugated amph-NPs (#1) and unmodified amph-NPs incubated with antibodies (#2). FIG. 1C shows quantitative analysis of SDS-PAGE results. FIG. 1D. ELISA showed anti-CD8 ADNCs remain functional and specific to CD8 antigen. FIG. 1E shows SDS-PAGE image of anti-CD8 single domain V_{H}H conjugated amph-NPs (#1), unmodified amph-NPs incubated with V_{H}H (#2), and unmodified amph-NPs alone (#3).

FIG. 2A shows an illustration of small-molecule loading into nanoparticle organic outer shell of antibody drug nanocarriers (ADNCs). FIG. 2B shows that drug loading efficiencies of bare gold nanoparticle vs. targeted-gold nanoparticle are similar. FIG. 2C shows that when TGFbeta inhibitor SB525334 is loaded onto amphiphilic gold nanoparticles, drug concentration dramatically increased in CD8+ T cells compared to free drug at the same incubation concentration in vitro. Free drug at 10-fold higher dose still resulted in lower drug/cell ratio compared to drug-loaded nanoparticles.

FIGS. 3A-3B show quantification of the number of gold nanoparticles per cell using Cytometer by Time-of-Flight (CyTOF) at 1 hour post-incubation of splenocytes with anti-CD8 conjugates and free nanoparticles (sans antibodies). FIG. 3A shows that splenar CD8⁺ T cell marker intensity decreased in the group which cells were pretreated with anti-CD8 antibody conjugated gold nanoparticles, suggesting receptor-mediated particle delivery occurred. FIG. 3B shows CD8⁺ T cells internalized 9.2-fold more gold nanoparticles than particles delivered freely without antibodies conjugated.

FIG. 4 shows that anti-CD8 conjugated gold nanoparticles enhance particle delivery to CD8⁺ T cells in the blood and spleen 24 hours post-intravenous injection.

FIG. 5 is an illustration of possible drug release mechanisms once ADNCs reach target cells.

FIGS. 6A-6C. TEM images of CD8+ T cells incubated with whole antibody conjugated amph-NPs showed that Ab-NPs were bound to surface receptors, followed by receptor-mediated endocytosis and intracellular membrane penetration. Cells were incubated with NPs for one hour and either fixed immediately (FIG. 6A) or incubated in serum-containing media (without NPs) for another 4 hours (FIG. 6B) or 24 hours (FIG. 6C) to allow for NP internalization and dispersion.

FIGS. 7A-7D show V_{H}H-conjugated amph-NP biodistribution in vivo (mice) 24 hours post tail vein intravenous injection. Quantification of number of NPs or V_{H}H-NPs in lymphocytes (FIG. 7A) and myeloid cells (FIG. 7B) isolated from the blood 24 hours post intravenous injection. Representative plots (FIG. 7C) and quantitative analysis (FIG. 7D) of the percentage of NP+ cells from mice receiving PBS, free NPs or V_{H}H-NPs. (N=3 per group, Two-way ANOVA with Tukey's multiple comparison test **** p<0.0001).

FIG. 8A-8E show that TGFβi delivered via V_{H}H-conjugated amph-NP drug delivery platform significantly augmented endogenous CD8+ T cell vaccine responses. FIG. 8A. Timeline of Trp1, Trp2, and gp100 trivalent vaccines and TGFβi administration in a B16F10 subcutaneous tumor model. FIG. 8B. Representative FACS plots of antigen specific CD8+ T cell responses. FIG. 8C. Quantitative analysis of percent of IFNg+ of total CD8+ T cells. (N=5 per group, one-way ANOVA with Tukey's multiple comparisons, ** p=0.0049). FIG. 8D. Representative FACS plots of polyfunctional CD8+ T cells. FIG. 8E. Quantitative analysis of percent of IFNg+TNFa+ of total CD8+ T cells. (N=5, One-way ANOVA with Tukey's multiple comparisons, * p=0.0393)

FIG. 9A-9B show mean fluorescence intensity (MFI) of PE-labled IFNg in ex vivo antigen stimulated CD8+ T cells. FIG. 9A. Representative FACS plot of MFI of IFNg 20 days post tumor inoculation. FIG. 9B. Quantitative analysis of MFI. (N=5, unpaired t test two-tailed p value, groups were compared to the VAX alone group, *** p=0.0009, ** p=0.0029, * p=0.0352).

### DETAILED DESCRIPTION OF CERTAIN EMBODIMENTS

The present invention provides nanoparticle-targeting agent conjugates (“conjugates” herein), and pharmaceutical compositions (i.e., formulations) thereof, which can be used in the delivery of therapeutic agents to cells and/or target tissues. Additionally, provided herein are method of preparing nanoparticle-targeting agent conjugates. The present invention also provides methods for delivering therapeutic agents to cells and target tissues, and methods of treating and/or preventing a disease (e.g., proliferative disease, autoimmune disease, infectious disease) in a subject. In certain embodiments, the methods comprise contacting a cell, target tissue, or biological sample with, or administering to a subject, a conjugate described herein, or a pharmaceutical composition thereof. Furthermore, the present invention provides kits (i.e., pharmaceutical packs) comprising the conjugates and compositions provided herein. In general, the particle-targeting agent conjugates provided herein provide a platform for directing and delivering enhanced payloads of therapeutic agents (e.g., small molecule drugs) to target cells. Targeted delivery may reduce the amount of therapeutic agent required for administration, as well as reduce off-target effects of the agent. Further, conjugates of the present invention can be used to expand the diversity of therapeutic agents (e.g., small molecule drugs) that can be delivered with the help of targeting agents, especially those therapeutic agents that are hydrophobic and cannot be delivered efficiently otherwise.

### Conjugates

One aspect of the present invention relates to nanoparticle-targeting agent conjugates (“conjugates”) which can be used in the delivery of therapeutic agents to a targeted cells or tissues. In certain embodiments, a conjugate of the present invention comprises one or more nanoparticles covalently linked to a targeting agent, wherein:

each nanoparticle comprises an organic outer shell;

one or more therapeutic agents are associated with the organic outer shell of each nanoparticle; and

each nanoparticle is less than approximately 10 nm in diameter.

The conjugates described herein comprise one or more nanoparticles per targeting agent. For instance, in certain embodiments, the conjugate comprises one nanoparticle per targeting agent (e.g., 1:1 nanoparticle-targeting agent ratio). In certain embodiments, the conjugate comprises two nanoparticles per targeting agent (e.g., 2:1 nanoparticle-targeting agent ratio). In certain embodiments, the ratio is greater than 2:1 nanoparticle-targeting agent. For example, when the targeting agent is an antibody, the conjugate consists of one nanoparticle covalently linked to one antibody. In another embodiment, the conjugate consists of two nanoparticles covalently linked to one antibody. However, more than two nanoparticles may be linked to a targeting agent (e.g., to an antibody or fragment thereof). In certain embodiments, the conjugate comprises no more than one targeting agent per nanoparticle.

The nanoparticle portion of the conjugate may be any type of particle. In certain embodiments, the nanoparticle is a metal nanoparticle (e.g., a transition metal nanoparticle). Examples of metal nanoparticles include, but are not limited to, gold, silver, copper, platinum, palladium, ruthenium, rhenium, iron, and nickel nanoparticles. The nanoparticle may comprise a single metal or a mixture of metals. In certain embodiments, the nanoparticle is a gold nanoparticle (AuNP). In certain embodiments, the nanoparticle comprises gold and one or more other materials. In certain embodiments, the nanoparticle comprises gold and one or more other metals.

In certain embodiments, provided herein is a gold nanoparticle (AuNP)-targeting agent conjugate comprising one or more gold nanoparticles covalently linked to a targeting agent; wherein:

the gold nanoparticle comprises an organic outer shell;

one or more therapeutic agents are associated with the organic outer shell of the gold nanoparticle; and

the gold nanoparticle is less than approximately 10 nm in diameter.

Conjugates of the present invention comprise a targeting agent covalently linked to one or more nanoparticles. The term “targeting agent” refers to any agent (e.g., molecule or substance) that targets a specific protein, cell, tissue, specific groups of proteins, cells, or tissues. In certain embodiments, the targeting agent is an antibody, peptide, protein, carbohydrate, nucleic acid, receptor ligand, small molecule, aptamer, receptor, particle, carbon nanostructure (e.g., carbon nanotube); or any fragment thereof. Other examples of targeting agents can be found in the art. In certain embodiments, the targeting agent is a protein.

In certain embodiments, the targeting agent is an antibody. In certain embodiments, the targeting agent is an antibody fragment. As described herein, targeting agents include, but are not limited to, polyclonal and monoclonal antibodies, antigen-binding fragments thereof (e.g., Fab, Fab′, F(ab′)₂, Fv), single chain antibodies (scFv), mutants thereof, fusion proteins comprising antibody portions, humanized antibodies, chimeric antibodies, diabodies, linear antibodies, single chain antibodies, multispecific antibodies (e.g., bispecific antibodies), single domain antibodies (sdAb), and any other modified configuration of the immunoglobulin molecule that comprises an antigen recognition site, including glycosylation variants of antibodies, amino acid sequence variants of antibodies, and covalently modified antibodies. As described herein, the antibody can be synthetic (e.g., recombinant) or animal-derived, wild-type (e.g., naturally occurring), or modified. In certain embodiments, the antibody is a humanized antibody. In certain embodiments, the antibody is a chimeric antibody. In certain embodiments, the antibody is a monoclonal antibody. In certain embodiments, the antibody is a polyclonal antibody. In certain embodiments, the targeting agent is an antibody fragment. In certain embodiments, the targeting agent is a IgD, IgE, IgG, IgA, or IgM antibody, or subclass thereof, or fragment thereof. In certain embodiments, the targeting agent is an IgG antibody. In certain embodiments, the targeting agent is an IgG antibody fragment. In certain embodiments, the targeting agent is a single domain antibody. In certain embodiments, the targeting agent is a single domain V_{H}H antibody or a fragment thereof. Other examples of antibodies and fragments thereof are provided in the Definitions.

The targeting agent may be an antibody for any antigen described herein. In certain embodiment, the antibody targets a cell. In certain embodiments, the antibody targets T cells. The antibody may target cells (e.g., T cells) by targeting and binding an antigen expressed in or on the cell, e.g., on the surface of the cell. In certain embodiments, the targeting agent is an antibody for a cluster of differentiation (CD) antigen (e.g., CD8). In certain embodiments, the targeting agent is an antibody for CD8 (i.e., “anti-CD8” antibody). Antibodies for CD8 recognize the CD8 antigen (e.g., human CD8 antigen) which is expressed on cytotoxic T cells (e.g., CD8⁺ T cells), thymocytes, and certain NK cells. In certain embodiments, the targeting agent is an anti-CD8 antibody that targets CD8⁺ T cells.

In certain embodiments, the targeting agent is an anti-CD8 IgG antibody, or a fragment thereof. In certain embodiments, the targeting agent is a IgD, IgE, IgG, IgA, or IgM anti-CD8 antibody, or a member of a subclass thereof. In certain embodiments, the targeting agent is a single domain anti-CD8 antibody. In certain embodiments, the targeting agent is a single domain V_{H}H antibody, or a fragment thereof. In certain embodiments, the targeting agent is a single domain anti-CD8 V_{H}H antibody, or a fragment thereof.

In certain embodiments, provided herein are gold nanoparticle (AuNP)-antibody conjugates comprising one or more gold nanoparticles covalently linked to an antibody, or a fragment thereof; wherein:

the gold nanoparticle comprises an organic outer shell;

one or more therapeutic agents are associated with the organic outer shell of the gold nanoparticle; and

the gold nanoparticle is smaller in size than the antibody (e.g., the AuNP is less than 10 nm in diameter).

In certain embodiments, provided herein are gold nanoparticle (AuNP)-antibody conjugates comprising one or more gold nanoparticles covalently linked to an antibody, or a fragment thereof; wherein:

the gold nanoparticle comprises an organic outer shell;

one or more therapeutic agents are associated with the organic outer shell of the gold nanoparticle; and

the gold nanoparticle is less than approximately 10 nm in diameter.

In embodiments wherein the targeting agent is an antibody, the one or more nanoparticles (e.g., gold nanoparticles) may be attached at any position on the antibody. In certain embodiments, the one or more nanoparticles are covalently linked to the antibody through the hinge region of the antibody. Conjugation through the hinge region of the antibody may be advantageous as it may allow the antibody to maintain its natural function and/or pharmacokinetics. In certain embodiments, the one or more nanoparticles are covalently linked through thiols (e.g., free cysteines) on the antibody. In certain embodiments, the one or more nanoparticles are covalently linked through amines (e.g., lysine residues) on the antibody. As described herein, disulfide bonds of an antibody can be reduced to expose free cysteines, which can be used as reactive moieties for conjugation to nanoparticles.

The conjugates provided herein comprise nanoparticles (e.g., gold nanoparticles) which are preferably 10 nm or less in diameter. In certain embodiments, the nanoparticles are less than 10 nm in diameter. The nanoparticles may be less than 10 nm, less than 9 nm, less than 8 nm, less than 7 nm, less than 6 nm, less than 5 nm, less than 4 nm, less than 3 nm, less than 2 nm, or less than 1 nm in diameter. In certain embodiments, the nanoparticles are less than 5 nm in diameter. In certain embodiments, the nanoparticles are from 1-10 nm, inclusive. In certain embodiments, the nanoparticles are from 2-8 nm, inclusive. In certain embodiments, the nanoparticles are from 2-6 nm, inclusive. In certain embodiments, the nanoparticles are from 2-4 nm, inclusive. In certain embodiments, the nanoparticles are about 3 nm in diameter. In certain embodiments, each nanoparticle is smaller in size than the targeting agent. For example, when the targeting agent is an antibody, each nanoparticle conjugated to the antibody is smaller in size than the antibody. For example, in certain embodiments, one or more nanoparticles less than 10 nm in diameter are conjugated to an antibody that is 10-20 nm. In a particular embodiment, one or more nanoparticles about 3 nm in diameter are conjugated to an antibody that is 10-20 nm. In certain embodiments, the nanoparticles being small in size relative to the targeting agent (e.g., antibody) is important to the targeting agent maintaining its natural function and/or pharmacokinetic properties.

Conjugates provided herein comprise nanoparticles (e.g., gold nanoparticles) with an organic outer shell. The “organic outer shell” is an outer layer of the nanoparticle that is comprised of organic (e.g., carbon-containing) moieties. The organic groups or moieties can be organic ligands that are associated with (i.e., bound to) the outer surface of the nanoparticles. In certain embodiments, the organic outer shell is an amphiphilic outer shell. The term “amphiphilic” refers to a substance that possesses both hydrophilic and hydrophobic properties. For example, in certain embodiments, the amphiphilic outer shell of a nanoparticle described herein comprises hydrophobic moieties as well as hydrophilic moieties. “Hydrophilic” and “hydrophobic” are defined herein. In certain embodiment, the outer shell comprises hydrophobic groups, such as unsubstituted aliphatic groups (e.g., unsubstituted, branched or unbranched alkyl groups; unsubstituted, branched or unbranched alkenyl groups; unsubstituted, branched or unbranched alkynyl groups; fluorine-substituted alkyl groups). In certain embodiments, the outer shell comprises hydrophilic groups, such as substituted aliphatic groups (e.g., substituted alkyl groups). As described herein, hydrophilic moieties are moieties comprising hydrogen bond donors groups and/or hydrogen bond acceptor groups (e.g., hydroxyl, amino, thio, sulfonate, sulfinate, carbonyl, phosphate, oxo groups, charged groups, and other substituents described herein). The organic outer shell of a nanoparticle may comprise polymeric ligands (e.g., polyethylene glycol (PEG)-containing ligands such as HS-PEG, HS-PEG-SO₃H, HS-PEG-NH₂, HS-PEG-OH, HS-PEG-CO₂H, HO-PEG-CO₂H, etc.)

In certain embodiments, the organic outer shell of the nanoparticle is made up of ligands comprising alkylsulfonate groups, unsubstituted alkyl groups, alkylamino groups, or any combination thereof. These ligands include, but are not limited to, compounds of the structure HS-alkyl, HS-alkyl-SO₃, HS-alkyl-NH₂, HS-alkyl-OH, HS-alkyl-CO₂H, etc. An example of an alkylsulfonate-containing ligand is 11-mercapto-1-undecanesulfonate (MUS). An example of an unsubstituted alkyl-containing ligand is octanethiol (OT). An example of an alkylamino-containing ligand is 11-amino-1-undcanethiol. Each of these specific compounds comprise a thiol group on one end, which is thought to coordinate to the outer sphere of the nanoparticle (e.g., when the nanoparticle is a metal nanoparticle such as a gold nanoparticle), thereby forming the organic outer shell of the nanoparticle. In a particular embodiment, the outer shell comprises 11-mercapto-1-undecanesulfonate (MUS), octanethiol (OT), or 11-amino-1-undcanethiol. Structures of these ligands are as follows:

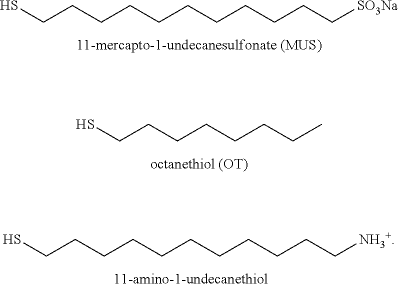

Examples of nanoparticles with organic outer shells, and ligands useful in the preparation thereof, can be found in, e.g., Rotello et al. Adv Drug Deliv Rev. 2012, 64, 200-216; and

references cited therein, all of which are incorporated herein by reference. Examples of nanoparticles with organic outer shells comprised of MUS and OT ligands can also be found in the literature. See, e.g., Irvine et al. Nature Materials 2008, 7, 588-595; Irvine et al. Nano Letters 2013, 13, 4060-4067; and references cited therein; all of which are incorporated herein by reference.

In certain embodiments, provided herein are gold nanoparticle (AuNP)-antibody conjugates comprising one or more gold nanoparticles covalently linked to an antibody, or a fragment thereof; wherein:

the gold nanoparticle comprises an amphiphilic outer shell;

one or more therapeutic agents are associated with the amphiphilic outer shell of the gold nanoparticle; and

the gold nanoparticle is less than approximately 10 nm in diameter.

The conjugates described herein comprise one or more therapeutic agents associated with the organic outer shell of each nanoparticle. The term “associated with,” when used with respect to two or more agents or components, means that the agents or components are physically associated or connected with one another, either directly or via one or more linking groups, to form a structure that is sufficiently stable so that the moieties remain physically associated for a desired period of time under certain conditions (e.g., ambient conditions, physiological conditions). The moieties can be associated by covalent or non-covalent interactions. In some instances, moieties are connected by covalent bonds. In some instances the moieties are connected through a linker (e.g., a cleavable linker, such as a hydrolysable linker). In some instances, moieties are connected by non-covalent interactions (e.g., ionic bonds, van der Waals forced, hydrophobic interactions, hydrophilic interactions, hydrogen bonding, or any combination thereof). In some embodiments, a sufficient number of weaker interactions can provide sufficient stability for moieties to remain physically associated under a variety of different conditions. In certain embodiments, the one or more therapeutic agents are adsorbed on the surface of the one or more nanoparticles. The terms “adsorbed,” “adsorption,” and the like refer to the adhesion of molecules to a surface. In certain embodiments, the organic outer shell of the nanoparticle includes hydrophobic shell vacancies (e.g., hydrophobic pockets) capable of adsorbing the one or more therapeutic agents onto the outer shell of the nanoparticle.

In certain embodiments, a conjugate comprises more than four (4) molecules of the therapeutic agent associated with the outer shell of a nanoparticle. In certain embodiments, a conjugate comprises more than 5, 10, 20, 30, 40, or 50 molecules of a therapeutic agent per gold nanoparticle. In certain embodiments, a conjugate comprises more than 50 molecules of a therapeutic agent. In certain embodiment, a conjugate comprises 50-200 molecules of a therapeutic agent. In certain embodiments, a conjugate comprises more than 100, 200, 300, 400, 500, or 1,000 molecules of a therapeutic agent. In certain embodiments, a conjugate comprises more than 200 molecules of a therapeutic agent. In certain embodiments, a conjugate comprises more than 1,000 molecules of a therapeutic agent.

The one or more therapeutic agents associated with the organic outer shell of the nanoparticles can be any therapeutic agent. Classes of therapeutic agents include, but are not limited to, small molecules, peptides, proteins, polymers, carbohydrates, and nucleic acids. In certain embodiments, the one or more therapeutic agents are small molecules (e.g., small molecule drugs). Any small molecule drug can be delivered using the conjugates described herein. In certain embodiments, the one or more therapeutic agents are selected from anti-proliferative agents, immunomodulators, and antibiotics. In certain embodiments, the one or more therapeutic agents are anti-proliferative agents (e.g., anti-cancer agents such as anti-cancer small molecules). Examples of anti-proliferative agents are provided herein. In certain embodiments, the therapeutic agent is a pharmaceutically acceptable salt of a therapeutic agent. The therapeutic agents associated with the nanoparticle conjugate may be a single therapeutic agent, or a combination of two or more different therapeutic agents. In certain embodiments, there are 2, 3, 4, or more different therapeutic agents associated with the nanoparticle. As described herein, conjugates of the present invention can be used to delivery therapeutic agents that are otherwise difficult to deliver to targets. In certain embodiments, one or more of the therapeutic agents have poor solubility. In certain embodiments, one or more of the therapeutic agents have a narrow therapeutic window. In certain embodiments, one or more of the therapeutic agents have exceptionally rapid clearance in vivo.

In certain embodiments, provided herein are gold nanoparticle (AuNP)-antibody conjugates comprising one or more gold nanoparticles covalently linked to an antibody, or a fragment thereof; wherein:

the gold nanoparticle comprises an amphiphilic outer shell;

one or more small molecule therapeutic agents are associated with the amphiphilic outer shell of the gold nanoparticle, wherein 50 or more (e.g., 50-200, or more than 200) molecules of the therapeutic agent are associated with the outer shell of each gold nanoparticle; and

the gold nanoparticle is less than approximately 10 nm in diameter.

Conjugates provided herein comprise one or more nanoparticles (e.g., gold nanoparticles) covalently linked to a targeting agent. The one or more nanoparticles can be linked to the targeting agent via any covalent linkage, e.g., via a covalent bond or a linker. In certain embodiments, the one or more nanoparticles are linked to the targeting agent via a covalent bond. In certain embodiments, the one or more nanoparticles are linked to the targeting agent via a linker. Examples of these particle-targeting agent linkages can be found in US Patent Publications, US 2013/0315834, published Nov. 28, 2013, and US 2009/0098574, published Apr. 16, 2009, each of which is incorporated herein by reference. The linker can be of any length, can include carbon atoms and/or heteroatoms, can include linear and/or cyclic moieties, can be branched or unbranched, and can be substituted or unsubstituted. In certain embodiments, the linker comprises a polymeric material (e.g., a peptidic linker or polyethylene glycol (PEG)). In certain embodiments, the one or more nanoparticles are conjugated to the targeting agent via click chemistry, and therefore the linker comprises a moiety derived from a click chemistry reaction (e.g., triazole, diazole, diazine, sulfide bond, maleimide ring, succinimide ring, ester, amide). In certain embodiments, a crosslinking reagent is used to conjugate the one or more nanoparticles to the targeting agent, and therefore part of the crosslinking reagent is present in the linker. “Crosslinking reagent” is defined herein, and examples of crosslinking reagents are provided herein.

For example, in certain embodiments, a crosslinking reagent such as m-maleimidobenzoyl-N-hydroxysulfosuccinimide ester (MBS) or sulfo-(m-maleimidobenzoyl-N-hydroxysulfosuccinimide ester) (Sulfo-MBS) is used, and the linking moiety comprises a group corresponding to one of the following formulae:

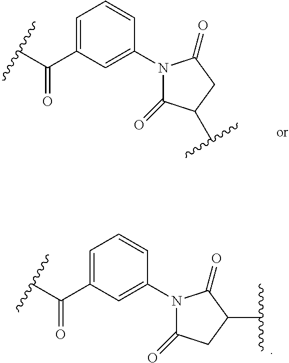 [SMILES: *C(=O)c1cccc(N2C(=O)CC(*)C2=O)c1 ; *C(=O)c1cccc(N2C(=O)CC(*)C2=O)c1]

In certain embodiments, the targeting agent is covalently linked to the one or more nanoparticles through the organic outer shells of the one or more nanoparticles. For example, the targeting agent may be linked to the one or more nanoparticles via reactive moieties on the organic outer shell of the nanoparticles (e.g., hydroxyls, amines, thiols). In a particular embodiment, the targeting agent is linked to the one or more nanoparticles via amines on the outer shell of the nanoparticles (e.g., via 11-amino-1-undcanethiol ligands on the nanoparticles). Likewise, the one or more nanoparticles can be linked to the targeting agent via reactive moieties on the targeting agent. In instances wherein the targeting agent is an antibody, the one or more nanoparticles can be linked to the antibody via thiols on the antibody.

### Methods for Preparing Conjugates

In certain embodiments, methods of preparing nanoparticle-targeting agent conjugates first involves providing a nanoparticle with an organic outer shell. Nanoparticles can be prepared according to methods known in the art, and currently there are methods for preparing nanoparticles (e.g., gold nanoparticles) with organic (e.g., amphiphilic) outer shells. See, e.g., Rotello et al. Adv Drug Deliv Review. 2012 64, 200-216; Mirkin et al. Angew Chem Int Ed Engl 2010, 49, 3280-3294; Singh et al. Nanomaterials 2011, 1, 31-63; Irvine et al. Nature Materials 2008, 7, 588-595; Irvine et al. Nano Letters 2013, 13, 4060-4067; and references cited therein, all of which are incorporated herein by reference.

The present invention provides methods of preparing the nanoparticle-targeting agent conjugates described herein. Any bond-forming reaction can be used to conjugate a nanoparticle (e.g., gold nanoparticle) to a targeting agent to form a nanoparticle-targeting agent conjugate of the present invention. Any methods known in the art of bioconjugation can be used (e.g., click chemistry reactions). For example, the nanoparticle may comprise a click chemistry handle on its outer shell, which can react with a click chemistry handle on a targeting agent, thereby covalently linking the nanoparticle with the targeting agent. In certain embodiments, a crosslinking reagent is used to conjugate the nanoparticle to the targeting agent.

“Crosslinking reagent” refers to a compound that comprises two or more reactive moieties. A crosslinking reagent is capable of forming a crosslink between two or more agents as follows: a reactive group on a first agent reacts and forms a covalent bond with a reactive moiety on the crosslinking agent, and a reactive group on a second agent reacts and forms a covalent bond with another reactive moiety on the crosslinking agent, thereby associating the first and second agents to one another via a crosslink. For example, a crosslinking reagent may be a molecule comprising a maleimide group and an ester group, both of which are reactive moieties. In certain embodiments, the maleimide group can react with a reactive moiety (e.g., a thiol) on one component (e.g., targeting agent, therapeutic agent), and the ester group could react with a reactive moiety (e.g., an amine) on the other component (e.g., targeting agent, therapeutic agent).

Examples of crosslinking reagents useful in the present invention include, but are not limited to, N-succinimidyl-3-[2-pyridyldithio]-propionamido (SPDP), succinimidyl-6-(3-[2-pyridyldithio]-propionamido)hexanoate (LC-SPDP), 4-succinimidyloxycarbonyl-methyl-α-[2-pyridyldithio]toluene (SMPT), 4-sulfosuccinimidyl-6-methyl-α-[2-pyridyldithio)toluamido]hexanoate) (Sulfo-LC-SMPT), succinimidyl 4-[N-maleimidomethyl]cyclohexane-1-carboxylate (SMCC), succinimidyl 4-[N-maleimidomethyl]cyclohexane-1-carboxy-[6-amidocaproate] (Sulfo-SMCC), m-maleimidobenzoyl-N-hydroxysuccinimide ester (MBS), m-maleimidobenzoyl-N-hydroxysulfosuccinimide ester (Sulfo-MBS), succinimidyl-4-[p-maleimidophenyl]butyrate (SMPB), sulfosuccinimidyl-4-[p-maleimidophenyl]butyrate (Sulfo-SMPB), N-[γ-maleimidobutyryloxy]succinimide ester (GMBS), N-[γ-maleimidobutyryloxy]sulfosuccinimide ester (Sulfo-GMBS), N-ε-maleimidocaproyloxy]succinimide ester (EMCS), N-ε-maleimidocaproyloxy]sulfosuccinimide ester (Sulfo-EMCS), N-succinimidyl S-acetyl(thiotetraethylene glycol), (1,4-bis-maleimidobutane (BMB), 1,4-bis-maleimidyl-2,3-dihydroxybutane (BMDB), bis-maleimidohexane (BMH), dimethyl pimelimidate (DMP), and bis[sulfosuccinimidyl]suberate (BS³).

A method of preparing a nanoparticle conjugate described herein includes the steps of: (a) providing a nanoparticle comprising an organic outer shell, wherein the organic outer shell comprises one or more reactive moieties; (b) contacting the nanoparticle provided in step (a) with a crosslinking reagent, thereby forming a covalent bond between a reactive moiety on the outer shell of the nanoparticle and the crosslinking reagent; (c) contacting the conjugate formed in step (b) with a targeting agent comprising one or more reactive moieties, thereby forming a crosslink between the targeting agent and the nanoparticle. The method of may further comprise a step of contacting the conjugate formed in step (c) with one or more therapeutic agents, thereby associating them with (e.g., absorbing them onto) the outer shell of the nanoparticle.

In a particular embodiment, the conjugate is a AuNP-antibody conjugate, and the method of preparing the comprises the steps of: (a) providing a gold nanoparticle comprising an organic outer shell, wherein the organic outer shell comprises one or more reactive moieties; (b) contacting the gold nanoparticle provided in step (a) with a crosslinking reagent, thereby forming a covalent bond between a reactive moiety on the outer shell of the nanoparticle and the crosslinking reagent; (c) contacting the conjugate formed in step (b) with an antibody comprising one or more reactive moieties, thereby forming a crosslink between the antibody and the gold nanoparticle. The method of may further comprise a step of contacting the conjugate formed in step (c) with one or more therapeutic agents, thereby associating them with (e.g., absorbing them onto) the outer shell of the gold nanoparticle. In certain embodiments, the reactive moiety on the organic outer shell of the nanoparticle is an amine (e.g., —NH₂). In certain embodiments, the reactive moiety on the organic outer shell of the nanoparticle is a thiol (e.g., —SH). In certain embodiments, the reactive moiety on targeting agent is an amine (e.g., —NH₂). In certain embodiments, the reactive moiety on the targeting agent is a thiol (e.g., —SH). In certain embodiments, the targeting agent is an antibody and the reactive moiety on the antibody is a thiol (e.g., —SH). In certain embodiments, the thiol is located in the hinge region of the antibody.

In certain embodiments, the crosslinking reagent comprises a maleimide group and an ester group. In certain embodiments, the crosslinking reagent is MBS or Sulfo-MBS. In a particular embodiment, the crosslinking reagent is Sulfo-MBS.

### Pharmaceutical Compositions, Kits, and Administration

The present disclosure provides pharmaceutical compositions (e.g., formulations) comprising a conjugate described herein, and optionally a pharmaceutically acceptable carrier. In certain embodiments, the conjugate described herein is provided in an effective amount in the pharmaceutical composition. In certain embodiments, the effective amount is a therapeutically effective amount. In certain embodiments, the effective amount is a prophylactically effective amount. In certain embodiments, the effective amount is an amount effective for treating or preventing a proliferative disease (e.g., cancer) in a subject in need thereof. In certain embodiments, the effective amount is an amount effective for treating and/or preventing an autoimmune disease in a subject in need thereof. In certain embodiments, the effective amount is an amount effective for treating and/or preventing an infectious disease in a subject in need thereof. In certain embodiments, the effective amount is an amount effective for reducing the risk of developing a disease (e.g., proliferative disease, autoimmune disease, infectious disease, inflammatory disease) in a subject in need thereof.

Compositions described herein can be prepared by any method known in the art of pharmacology. In general, such preparatory methods include bringing the conjugate into association with a carrier or excipient, one or more therapeutic agents, and/or one or more other accessory ingredients, and then, if necessary and/or desirable, diluting, shaping, and/or packaging the product into a desired single- or multi-dose unit.

Compositions can be prepared, packaged, and/or sold in bulk, as a single unit dose, and/or as a plurality of single unit doses. A “unit dose” is a discrete amount of the pharmaceutical composition comprising a predetermined amount of the conjugate. The amount of the conjugate is generally equal to the dosage of the conjugate which would be administered to a subject and/or a convenient fraction of such a dosage, such as one-half or one-third of such a dosage.

Relative amounts of the conjugate, the pharmaceutically acceptable excipient, and/or any additional ingredients in a pharmaceutical composition described herein will vary, depending upon the identity, size, and/or condition of the subject treated and further depending upon the route by which the composition is to be administered. The composition may comprise between 0.1% and 100% (w/w) conjugate. In certain embodiments, the composition comprises between about 0.1% and about 50% (w/w) conjugate.

Pharmaceutically acceptable excipients or carriers used in the manufacture of provided pharmaceutical compositions include inert diluents, dispersing and/or granulating agents, surface active agents and/or emulsifiers, disintegrating agents, binding agents, preservatives, buffering agents, lubricating agents, and/or oils. Excipients such as cocoa butter and suppository waxes, coloring agents, coating agents, sweetening, flavoring, and perfuming agents may also be present in the composition. In certain embodiments, the carrier or excipient is water. In certain embodiments, the carrier or excipient is a buffered aqueous solution.

Exemplary diluents include calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, and mixtures thereof.

Exemplary granulating and/or dispersing agents include potato starch, corn starch, tapioca starch, sodium starch glycolate, clays, alginic acid, guar gum, citrus pulp, agar, bentonite, cellulose, and wood products, natural sponge, cation-exchange resins, calcium carbonate, silicates, sodium carbonate, cross-linked poly(vinyl-pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (Veegum), sodium lauryl sulfate, quaternary ammonium compounds, and mixtures thereof.

Exemplary surface active agents and/or emulsifiers include natural emulsifiers (e.g., acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g., bentonite (aluminum silicate) and Veegum (magnesium aluminum silicate)), long chain amino acid derivatives, high molecular weight alcohols (e.g., stearyl alcohol, cetyl alcohol, oleyl alcohol, triacetin monostearate, ethylene glycol distearate, glyceryl monostearate, and propylene glycol monostearate, polyvinyl alcohol), carbomers (e.g., carboxy polymethylene, polyacrylic acid, acrylic acid polymer, and carboxyvinyl polymer), carrageenan, cellulosic derivatives (e.g., carboxymethylcellulose sodium, powdered cellulose, hydroxymethyl cellulose, hydroxypropyl cellulose, hydroxypropyl methylcellulose, methylcellulose), sorbitan fatty acid esters (e.g., polyoxyethylene sorbitan monolaurate (Tween® 20), polyoxyethylene sorbitan (Tween® 60), polyoxyethylene sorbitan monooleate (Tween® 80), sorbitan monopalmitate (Span® 40), sorbitan monostearate (Span® 60), sorbitan tristearate (Span® 65), glyceryl monooleate, sorbitan monooleate (Span® 80), polyoxyethylene esters (e.g., polyoxyethylene monostearate (Myrj® 45), polyoxyethylene hydrogenated castor oil, polyethoxylated castor oil, polyoxymethylene stearate, and Solutol®), sucrose fatty acid esters, polyethylene glycol fatty acid esters (e.g., Cremophor®), polyoxyethylene ethers, (e.g., polyoxyethylene lauryl ether (Brij® 30)), poly(vinyl-pyrrolidone), diethylene glycol monolaurate, triethanolamine oleate, sodium oleate, potassium oleate, ethyl oleate, oleic acid, ethyl laurate, sodium lauryl sulfate, Pluronic® F-68, poloxamer P-188, cetrimonium bromide, cetylpyridinium chloride, benzalkonium chloride, docusate sodium, and/or mixtures thereof.

Exemplary binding agents include starch (e.g., cornstarch and starch paste), gelatin, sugars (e.g., sucrose, glucose, dextrose, dextrin, molasses, lactose, lactitol, mannitol, etc.), natural and synthetic gums (e.g., acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, poly(vinyl-pyrrolidone), magnesium aluminum silicate (Veegum®), and larch arabogalactan), alginates, polyethylene oxide, polyethylene glycol, inorganic calcium salts, silicic acid, polymethacrylates, waxes, water, alcohol, and/or mixtures thereof.

Exemplary preservatives include antioxidants, chelating agents, antimicrobial preservatives, antifungal preservatives, antiprotozoan preservatives, alcohol preservatives, acidic preservatives, and other preservatives. In certain embodiments, the preservative is an antioxidant. In other embodiments, the preservative is a chelating agent.

Exemplary antioxidants include alpha tocopherol, ascorbic acid, ascorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and sodium sulfite.

Exemplary chelating agents include ethylenediaminetetraacetic acid (EDTA) and salts and hydrates thereof (e.g., sodium edetate, disodium edetate, trisodium edetate, calcium disodium edetate, dipotassium edetate, and the like), citric acid and salts and hydrates thereof (e.g., citric acid monohydrate), fumaric acid and salts and hydrates thereof, malic acid and salts and hydrates thereof, phosphoric acid and salts and hydrates thereof, and tartaric acid and salts and hydrates thereof. Exemplary antimicrobial preservatives include benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and thimerosal.

Exemplary antifungal preservatives include butyl paraben, methyl paraben, ethyl paraben, propyl paraben, benzoic acid, hydroxybenzoic acid, potassium benzoate, potassium sorbate, sodium benzoate, sodium propionate, and sorbic acid. Exemplary alcohol preservatives include ethanol, polyethylene glycol, phenol, phenolic compounds, bisphenol, chlorobutanol, hydroxybenzoate, and phenylethyl alcohol.

Exemplary acidic preservatives include vitamin A, vitamin C, vitamin E, beta-carotene, citric acid, acetic acid, dehydroacetic acid, ascorbic acid, sorbic acid, and phytic acid.

Other preservatives include tocopherol, tocopherol acetate, deteroxime mesylate, cetrimide, butylated hydroxyanisol (BHA), butylated hydroxytoluened (BHT), ethylenediamine, sodium lauryl sulfate (SLS), sodium lauryl ether sulfate (SLES), sodium bisulfite, sodium metabisulfite, potassium sulfite, potassium metabisulfite, Glydant® Plus, Phenonip®, methylparaben, Germall® 115, Germaben® II, Neolone®, Kathon®, and Euxyl®.

Exemplary buffering agents include citrate buffer solutions, acetate buffer solutions, phosphate buffer solutions, ammonium chloride, calcium carbonate, calcium chloride, calcium citrate, calcium glubionate, calcium gluceptate, calcium gluconate, D-gluconic acid, calcium glycerophosphate, calcium lactate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, magnesium hydroxide, aluminum hydroxide, alginic acid, pyrogen-free water, isotonic saline, Ringer's solution, ethyl alcohol, and mixtures thereof.

Exemplary lubricating agents include magnesium stearate, calcium stearate, stearic acid, silica, talc, malt, glyceryl behanate, hydrogenated vegetable oils, polyethylene glycol, sodium benzoate, sodium acetate, sodium chloride, leucine, magnesium lauryl sulfate, sodium lauryl sulfate, and mixtures thereof.

Exemplary natural oils include almond, apricot kernel, avocado, babassu, bergamot, black current seed, borage, cade, camomile, canola, caraway, carnauba, castor, cinnamon, cocoa butter, coconut, cod liver, coffee, corn, cotton seed, emu, eucalyptus, evening primrose, fish, flaxseed, geraniol, gourd, grape seed, hazel nut, hyssop, isopropyl myristate, jojoba, kukui nut, lavandin, lavender, lemon, litsea cubeba, macadamia nut, mallow, mango seed, meadowfoam seed, mink, nutmeg, olive, orange, orange roughy, palm, palm kernel, peach kernel, peanut, poppy seed, pumpkin seed, rapeseed, rice bran, rosemary, safflower, sandalwood, sasquana, savoury, sea buckthorn, sesame, shea butter, silicone, soybean, sunflower, tea tree, thistle, tsubaki, vetiver, walnut, and wheat germ oils. Exemplary synthetic oils include, but are not limited to, butyl stearate, caprylic triglyceride, capric triglyceride, cyclomethicone, diethyl sebacate, dimethicone 360, isopropyl myristate, mineral oil, octyldodecanol, oleyl alcohol, silicone oil, and mixtures thereof.

Liquid dosage forms for oral and parenteral administration include pharmaceutically acceptable emulsions, microemulsions, solutions, suspensions, syrups and elixirs. In addition to the active ingredients, the liquid dosage forms may comprise inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (e.g., cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can include adjuvants such as wetting agents, emulsifying and suspending agents, sweetening, flavoring, and perfuming agents. In certain embodiments for parenteral administration, the conjugates described herein are mixed with solubilizing agents such as Cremophor®, alcohols, oils, modified oils, glycols, polysorbates, cyclodextrins, polymers, and mixtures thereof.

Injectable preparations, for example, sterile injectable aqueous or oleaginous suspensions can be formulated according to the known art using suitable dispersing or wetting agents and suspending agents. The sterile injectable preparation can be a sterile injectable solution, suspension, or emulsion in a nontoxic parenterally acceptable diluent or solvent, for example, as a solution in 1,3-butanediol. Among the acceptable vehicles and solvents that can be employed are water, Ringer's solution, U.S.P., and isotonic sodium chloride solution. In addition, sterile, fixed oils are conventionally employed as a solvent or suspending medium. For this purpose any bland fixed oil can be employed including synthetic mono- or di-glycerides. In addition, fatty acids such as oleic acid are used in the preparation of injectables. The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

Compositions for rectal or vaginal administration are typically suppositories which can be prepared by mixing the conjugates described herein with suitable non-irritating excipients or carriers such as cocoa butter, polyethylene glycol, or a suppository wax which are solid at ambient temperature but liquid at body temperature and therefore melt in the rectum or vaginal cavity and release the active ingredient.

Where appropriate, the conjugates may be formulated in solid dosage forms for oral administration, including capsules, tablets, pills, powders, and granules. Solid compositions of a similar type can be employed as fillers in soft and hard-filled gelatin capsules using such excipients as lactose or milk sugar as well as high molecular weight polyethylene glycols and the like. The active ingredient can be in a micro-encapsulated form with one or more excipients as noted above. The solid dosage forms of tablets, dragees, capsules, pills, and granules can be prepared with coatings and shells such as enteric coatings, release controlling coatings, and other coatings well known in the pharmaceutical formulating art

Dosage forms for topical and/or transdermal administration of a conjugate described herein may include ointments, pastes, creams, lotions, gels, powders, solutions, sprays, inhalants, and/or patches. Generally, the active ingredient is admixed under sterile conditions with a pharmaceutically acceptable carrier or excipient and/or any needed preservatives and/or buffers as can be required. Additionally, the present disclosure contemplates the use of transdermal patches, which often have the added advantage of providing controlled delivery of an active ingredient to the body. Such dosage forms can be prepared, for example, by dissolving and/or dispensing the active ingredient in the proper medium. Alternatively or additionally, the rate can be controlled by either providing a rate controlling membrane and/or by dispersing the active ingredient in a polymer matrix and/or gel.

Suitable devices for use in delivering intradermal pharmaceutical compositions described herein include short needle devices. Intradermal compositions can be administered by devices which limit the effective penetration length of a needle into the skin. Alternatively or additionally, conventional syringes can be used in the classical mantoux method of intradermal administration. Jet injection devices which deliver liquid formulations to the dermis via a liquid jet injector and/or via a needle which pierces the stratum corneum and produces a jet which reaches the dermis are suitable. Ballistic powder/particle delivery devices which use compressed gas to accelerate the composition through the outer layers of the skin to the dermis are suitable.

Formulations suitable for topical administration include, but are not limited to, liquid and/or semi-liquid preparations such as liniments, lotions, oil-in-water and/or water-in-oil emulsions such as creams, ointments, and/or pastes, and/or solutions and/or suspensions. Topically administrable formulations may, for example, comprise from about 1% to about 10% (w/w) active ingredient, although the concentration of the active ingredient can be as high as the solubility limit of the active ingredient in the solvent. Formulations for topical administration may further comprise one or more of the additional ingredients described herein.

A composition described herein can be prepared, packaged, and/or sold in a formulation suitable for pulmonary administration via the buccal cavity. Pharmaceutical compositions described herein formulated for pulmonary delivery may provide the active ingredient in the form of droplets of a solution and/or suspension. Such formulations can be prepared, packaged, and/or sold as aqueous and/or dilute alcoholic solutions and/or suspensions, optionally sterile, comprising the active ingredient, and may conveniently be administered using any nebulization and/or atomization device. Such formulations may further comprise one or more additional ingredients including, but not limited to, a flavoring agent such as saccharin sodium, a volatile oil, a buffering agent, a surface active agent, and/or a preservative such as methylhydroxybenzoate. Formulations described herein as being useful for pulmonary delivery are useful for intranasal delivery of a pharmaceutical composition described herein.

A pharmaceutical composition described herein can be prepared, packaged, and/or sold in a formulation for ophthalmic administration. Such formulations may, for example, be in the form of eye drops including, for example, a 0.1-1.0% (w/w) solution and/or suspension of the active ingredient in an aqueous or oily liquid carrier or excipient. Such drops may further comprise buffering agents, salts, and/or one or more other of the additional ingredients described herein. Other opthalmically-administrable formulations which are useful include those which comprise the active ingredient in microcrystalline form and/or in a liposomal preparation. Ear drops and/or eye drops are also contemplated as being within the scope of this disclosure.

Although the descriptions of compositions provided herein are principally directed to pharmaceutical compositions which are suitable for administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and/or perform such modification with ordinary experimentation.

Conjugates provided herein are typically formulated in dosage unit form for ease of administration and uniformity of dosage. It will be understood, however, that the total daily usage of the compositions described herein will be decided by a physician within the scope of sound medical judgment. The specific therapeutically effective dose level for any particular subject or organism will depend upon a variety of factors including the disease being treated and the severity of the disorder; the activity of the specific active ingredient employed; the specific composition employed; the age, body weight, general health, sex, and diet of the subject; the time of administration, route of administration, and rate of excretion of the specific active ingredient employed; the duration of the treatment; drugs used in combination or coincidental with the specific active ingredient employed; and like factors well known in the medical arts.

The conjugates and compositions provided herein can be administered by any route, including enteral (e.g., oral), parenteral, intravenous, intramuscular, intra-arterial, intramedullary, intrathecal, subcutaneous, intraventricular, transdermal, interdermal, rectal, intravaginal, intraperitoneal, topical (as by powders, ointments, creams, and/or drops), mucosal, nasal, bucal, sublingual; by intratracheal instillation, bronchial instillation, and/or inhalation; and/or as an oral spray, nasal spray, and/or aerosol. Specifically contemplated routes intravenous administration (e.g., systemic intravenous injection), regional administration via blood and/or lymph supply, and/or direct administration to an affected site. In general, the most appropriate route of administration will depend upon a variety of factors including the nature of the agent (e.g., its stability in the environment of the gastrointestinal tract), and/or the condition of the subject (e.g., whether the subject is able to tolerate oral administration).

The exact amount of a conjugate required to achieve an effective amount will vary from subject to subject, depending, for example, on species, age, and general condition of a subject, severity of the side effects or disorder, identity of the particular conjugate, mode of administration, and the like. An effective amount may be included in a single dose (e.g., single oral dose) or multiple doses (e.g., multiple oral doses). In certain embodiments, when multiple doses are administered to a subject or applied to a tissue or cell, any two doses of the multiple doses include different or substantially the same amounts of a conjugate described herein. In certain embodiments, when multiple doses are administered to a subject or applied to a tissue or cell, the frequency of administering the multiple doses to the subject or applying the multiple doses to the tissue or cell is three doses a day, two doses a day, one dose a day, one dose every other day, one dose every third day, one dose every week, one dose every two weeks, one dose every three weeks, or one dose every four weeks. In certain embodiments, the frequency of administering the multiple doses to the subject or applying the multiple doses to the tissue or cell is one dose per day. In certain embodiments, the frequency of administering the multiple doses to the subject or applying the multiple doses to the tissue or cell is two doses per day. In certain embodiments, the frequency of administering the multiple doses to the subject or applying the multiple doses to the tissue or cell is three doses per day. In certain embodiments, when multiple doses are administered to a subject or applied to a tissue or cell, the duration between the first dose and last dose of the multiple doses is one day, two days, four days, one week, two weeks, three weeks, one month, two months, three months, four months, six months, nine months, one year, two years, three years, four years, five years, seven years, ten years, fifteen years, twenty years, or the lifetime of the subject, tissue, or cell. In certain embodiments, the duration between the first dose and last dose of the multiple doses is three months, six months, or one year. In certain embodiments, the duration between the first dose and last dose of the multiple doses is the lifetime of the subject, tissue, or cell.

Conjugates described herein are useful in the targeted delivery of one or more therapeutic agents (e.g., small molecule drugs) to a cell of a subject. As described herein, the therapeutic agents are associated with the organic outer shell of the nanoparticle. In certain embodiments, a dose (e.g., a single dose, or any dose of multiple doses) described herein includes independently between 0.1 ag and 1 ag, between 0.001 mg and 0.01 mg, between 0.01 mg and 0.1 mg, between 0.1 mg and 1 mg, between 1 mg and 3 mg, between 3 mg and 10 mg, between 10 mg and 30 mg, between 30 mg and 100 mg, between 100 mg and 300 mg, between 300 mg and 1,000 mg, or between 1 g and 10 g, inclusive, of a therapeutic agent (i.e., the total weight therapeutic agent, not including the rest of the conjugate (e.g., particle, targeting moiety, linker, etc.)). In certain embodiments, a dose described herein includes independently between 1 mg and 3 mg, inclusive, of a therapeutic agent. In certain embodiments, a dose described herein includes independently between 3 mg and 10 mg, inclusive, of a therapeutic agent. In certain embodiments, a dose described herein includes independently between 10 mg and 30 mg, inclusive, of a therapeutic agent. In certain embodiments, a dose described herein includes independently between 30 mg and 100 mg, inclusive, of a therapeutic agent.

In certain embodiments, an effective amount of a therapeutic agent (i.e., the weight therapeutic agent, not including the rest of the conjugate (e.g., particle, targeting moiety, linker, etc.)) for administration one or more times a day to a 70 kg adult human may comprise about 0.0001 mg to about 3000 mg, about 0.0001 mg to about 2000 mg, about 0.0001 mg to about 1000 mg, about 0.001 mg to about 1000 mg, about 0.01 mg to about 1000 mg, about 0.1 mg to about 1000 mg, about 1 mg to about 1000 mg, about 1 mg to about 100 mg, about 10 mg to about 1000 mg, or about 100 mg to about 1000 mg, of a therapeutic agent per unit dosage form.

In certain embodiments, the therapeutic agents described herein (i.e., the weight therapeutic agent, not including the rest of the conjugate (e.g., particle, targeting moiety, linker, etc.)) may be at dosage levels sufficient to deliver from about 0.001 mg/kg to about 100 mg/kg, from about 0.01 mg/kg to about 50 mg/kg, preferably from about 0.1 mg/kg to about 40 mg/kg, preferably from about 0.5 mg/kg to about 30 mg/kg, from about 0.01 mg/kg to about 10 mg/kg, from about 0.1 mg/kg to about 10 mg/kg, and more preferably from about 1 mg/kg to about 25 mg/kg, of subject body weight per day, one or more times a day, to obtain the desired therapeutic and/or prophylactic effect.

Dose ranges as described herein provide guidance for the administration of provided pharmaceutical compositions to an adult. The amount to be administered to, for example, a child or an adolescent can be determined by a medical practitioner or person skilled in the art and can be lower or the same as that administered to an adult.

A conjugate or composition, as described herein, can be administered in combination with one or more additional pharmaceutical agents (e.g., therapeutically and/or prophylactically active agents). The conjugates or compositions can be administered in combination with additional pharmaceutical agents that improve their activity (e.g., activity (e.g., potency and/or efficacy) in treating a disease in a subject in need thereof, in preventing a disease in a subject in need thereof, in reducing the risk to develop a disease in a subject in need thereof, and/or in inhibiting the activity of a protein kinase in a subject or cell), improve bioavailability, improve safety, reduce drug resistance, reduce and/or modify metabolism, inhibit excretion, and/or modify distribution in a subject or cell. It will also be appreciated that the therapy employed may achieve a desired effect for the same disorder, and/or it may achieve different effects. In certain embodiments, a pharmaceutical composition described herein including a conjugate described herein and an additional pharmaceutical agent shows a synergistic effect that is absent in a pharmaceutical composition including one of the conjugates and the additional pharmaceutical agent, but not both.

The conjugate or composition can be administered concurrently with, prior to, or subsequent to one or more additional pharmaceutical agents, which may be useful as, e.g., combination therapies. Pharmaceutical agents include therapeutically active agents. Pharmaceutical agents also include prophylactically active agents. Pharmaceutical agents include small organic molecules such as drug compounds (e.g., compounds approved for human or veterinary use by the U.S. Food and Drug Administration as provided in the Code of Federal Regulations (CFR)), peptides, proteins, carbohydrates, monosaccharides, oligosaccharides, polysaccharides, nucleoproteins, mucoproteins, lipoproteins, synthetic polypeptides or proteins, small molecules linked to proteins, glycoproteins, steroids, nucleic acids, DNAs, RNAs, nucleotides, nucleosides, oligonucleotides, antisense oligonucleotides, lipids, hormones, vitamins, and cells. In certain embodiments, the additional pharmaceutical agent is a pharmaceutical agent useful for treating and/or preventing a disease (e.g., proliferative disease, hematological disease, neurological disease, painful condition, psychiatric disorder, or metabolic disorder). Each additional pharmaceutical agent may be administered at a dose and/or on a time schedule determined for that pharmaceutical agent. The additional pharmaceutical agents may also be administered together with each other and/or with the conjugate or composition described herein in a single dose or administered separately in different doses. The particular combination to employ in a regimen will take into account compatibility of the conjugate described herein with the additional pharmaceutical agent(s) and/or the desired therapeutic and/or prophylactic effect to be achieved. In general, it is expected that the additional pharmaceutical agent(s) in combination be utilized at levels that do not exceed the levels at which they are utilized individually. In some embodiments, the levels utilized in combination will be lower than those utilized individually.

The additional pharmaceutical agents include, but are not limited to, anti-proliferative agents, anti-cancer agents, anti-angiogenesis agents, anti-inflammatory agents, immunosuppressants, anti-bacterial agents, anti-viral agents, cardiovascular agents, cholesterol-lowering agents, anti-diabetic agents, anti-allergic agents, contraceptive agents, and pain-relieving agents.

Also encompassed by the disclosure are kits (e.g., pharmaceutical packs). The kits provided may comprise a pharmaceutical composition or conjugate described herein and a container (e.g., a vial, ampule, bottle, syringe, and/or dispenser package, or other suitable container). In some embodiments, provided kits may optionally further include a second container comprising a pharmaceutical excipient for dilution or suspension of a pharmaceutical composition or conjugate described herein. In some embodiments, the pharmaceutical composition or conjugate described herein provided in the first container and the second container are combined to form one unit dosage form.

Thus, in one aspect, provided are kits including a first container comprising a conjugate or pharmaceutical composition described herein. In certain embodiments, the kits are useful for treating a disease (e.g., proliferative disease, autoimmune disease, infectious disease) in a subject in need thereof. In certain embodiments, the kits are useful for preventing a disease (e.g., proliferative disease, autoimmune disease, infectious disease) in a subject in need thereof. In certain embodiments, the kits are useful for reducing the risk of developing a disease (e.g., proliferative disease, autoimmune disease, infectious disease) in a subject in need thereof.

In certain embodiments, a kit described herein further includes instructions for using the kit. A kit described herein may also include information as required by a regulatory agency such as the U.S. Food and Drug Administration (FDA). In certain embodiments, the information included in the kits is prescribing information. In certain embodiments, the kits and instructions provide for treating a disease (e.g., proliferative disease, autoimmune disease, infectious disease) in a subject in need thereof. In certain embodiments, the kits and instructions provide for preventing a disease (e.g., proliferative disease, autoimmune disease, infectious disease) in a subject in need thereof. In certain embodiments, the kits and instructions provide for reducing the risk of developing a disease (e.g., proliferative disease, autoimmune disease, infectious disease) in a subject in need thereof. A kit described herein may include one or more additional pharmaceutical agents described herein as a separate composition.

### Methods of Treatment and Use

The present invention provides methods for delivering one or more therapeutic agents to a cell, the method comprising contacting the cell with a conjugate provided herein, or a pharmaceutical composition thereof. In certain embodiments, the one or more therapeutic agents are effectively delivered to the cytosol of the cell. In certain embodiments, the cell is contacted in vitro. In certain embodiments, the cell is contacted in vivo (e.g., in a subject). In certain embodiments, the cell is contacted ex vivo. In certain embodiments, the cell is a cancer cell (e.g., human cancer cell). In certain embodiments, the cell is an immune cell. In certain embodiments, the cell is a blood cell. In certain embodiments, the cell is an epithelial cell.

The present invention also provides uses of conjugates described herein, and pharmaceutical compositions thereof, for the delivery of one or more therapeutic agents to a cell. Further, provided herein are conjugates, and pharmaceutical compositions thereof, for use in delivering one or more therapeutic agents to a cell.

The use of nanoparticle-targeting agent conjugates described herein can confer improved delivery of the nanoparticles with their payload to target cells. In certain embodiments, a conjugate described herein confers delivery of the nanoparticle to a cell that is greater than, or least 2-fold, 3-fold, 4-fold, 5-fold, 6-fold, 7-fold, 8-fold, 9-fold, 10 fold, 20-fold, 50-fold, or 100-fold greater than the delivery of the Nanoparticle without the targeting agent conjugated thereto. According to the present invention, therapeutic agents are associated with the outer shells of the nanoparticles, and therefore conjugates described herein can confer improved delivery of the one or more therapeutic agents to cells. In certain embodiments, a conjugate described herein confers delivery of one or more therapeutic agents to a cell that is greater than, or at least 2-fold, 3-fold, 4-fold, 5-fold, 6-fold, 7-fold, 8-fold, 9-fold, 10 fold, 20-fold, 50-fold, or 100-fold greater than the delivery of the therapeutic agent, as compared with delivery of the therapeutic agent when it is not associated with the conjugate.

In certain embodiments, the methods described herein comprise contacting a biological sample with an effective amount of a conjugate described herein, or a pharmaceutical composition thereof. In certain embodiments, the biological sample is obtained from a subject. In certain embodiments, the methods described herein comprise contacting a target tissue with an effective amount of a conjugate described herein, or a pharmaceutical composition thereof.

The present invention also provides methods for treating a disease or condition in a subject, the methods comprising administering to the subject a conjugate of the present invention, or a pharmaceutical composition thereof. In certain embodiments, the disease or condition is a genetic disease, proliferative disease (e.g., cancer), a disease associated with angiogenesis, a neoplasm, inflammatory disease, autoimmune disease, liver disease, spleen disease, pulmonary disease, hematological disease, neurological disease, painful condition, psychiatric disorder, or metabolic disorder (e.g., a diabetic condition). In certain embodiments, the disease is a proliferative disease (e.g., cancer, autoimmune disease, inflammatory disease). In certain embodiments, the disease is cancer. In certain embodiments, the disease is an infectious disease.

The present invention also provides uses of conjugates, and pharmaceutical compositions thereof, for the manufacture of medicaments for the treatment and/or prevention of diseases or conditions (e.g., proliferative diseases such as cancer, autoimmune diseases, inflammatory diseases, infectious diseases). Further, the present invention provides conjugates, and pharmaceutical compositions thereof, for use in the treatment and/or prevention of diseases or conditions (e.g., proliferative diseases such as cancer, autoimmune diseases, inflammatory diseases, infectious diseases).

The methods provided herein comprise administering to a subject a conjugate described herein, or a pharmaceutical composition thereof. A “therapeutically effective amount” of a conjugate described herein is an amount sufficient to provide a therapeutic benefit in the treatment of a condition or to delay or minimize one or more symptoms associated with the condition. A therapeutically effective amount of an agent means an amount of an agent, alone or in combination with other therapies, which provides a therapeutic benefit in the treatment of the condition. The term “therapeutically effective amount” can encompass an amount that improves overall therapy, reduces or avoids symptoms, signs, or causes of the condition, and/or enhances the therapeutic efficacy of another therapeutic agent.

In certain embodiments, the methods described herein comprise administering to a subject a prophylactically effective amount of a conjugate provided herein, or a pharmaceutical composition thereof. A “prophylactically effective amount” of a conjugate described herein is an amount sufficient to prevent a condition, or one or more symptoms associated with the condition or prevent its recurrence. A prophylactically effective amount of an agent means an amount of an agent, alone or in combination with other agents, which provides a prophylactic benefit in the prevention of the condition. The term “prophylactically effective amount” can encompass an amount that improves overall prophylaxis or enhances the prophylactic efficacy of another prophylactic agent.

A conjugate provided herein may be administered concurrently with, prior to, or subsequent to, one or more additional therapeutic agents. In general, each agent will be administered at a dose and/or on a time schedule determined for that agent. It will further be appreciated that the additional therapeutically active agent utilized in this combination can be administered together in a single composition or administered separately in different compositions. The particular combination to employ in a regimen will take into account compatibility of the inventive conjugate with the additional therapeutically active agent and/or the desired therapeutic effect to be achieved. In general, it is expected that additional therapeutically active agents utilized in combination be utilized at levels that do not exceed the levels at which they are utilized individually. In some embodiments, the levels utilized in combination will be lower than those utilized individually. In certain embodiments, the additional therapeutic agent is an anti-proliferative agent. In certain embodiments, the anti-proliferative agent is an anti-cancer agent. Examples of anti-proliferative agents (e.g., anti-cancer agents) are provided herein.

In certain embodiments, the conjugates or pharmaceutical compositions described herein can be administered in combination with an anti-cancer therapy including, but not limited to, surgery, radiation therapy, transplantation (e.g., stem cell transplantation, bone marrow transplantation), immunotherapy, and chemotherapy.

In certain embodiments, the subject is an animal. The animal may be of either sex and may be at any stage of development. In certain embodiments, the subject described herein is a human. In certain embodiments, the subject is a non-human animal. In certain embodiments, the subject is a mammal. In certain embodiments, the subject is a non-human mammal. In certain embodiments, the subject is a domesticated animal, such as a dog, cat, cow, pig, horse, sheep, or goat. In certain embodiments, the subject is a companion animal, such as a dog or cat. In certain embodiments, the subject is a livestock animal, such as a cow, pig, horse, sheep, or goat. In certain embodiments, the subject is a zoo animal. In another embodiment, the subject is a research animal, such as a rodent (e.g., mouse, rat), dog, pig, or non-human primate. In certain embodiments, the animal is a genetically engineered animal. In certain embodiments, the animal is a transgenic animal (e.g., transgenic mice and transgenic pigs). In certain embodiments, the subject is a fish or reptile.

### Examples

These and other aspects of the present invention will be further appreciated upon consideration of the following Examples, which are intended to illustrate certain particular embodiments of the invention but are not intended to limit its scope, as defined by the claims.

### Chemical Conjugation of Nanoparticles to Antibodies

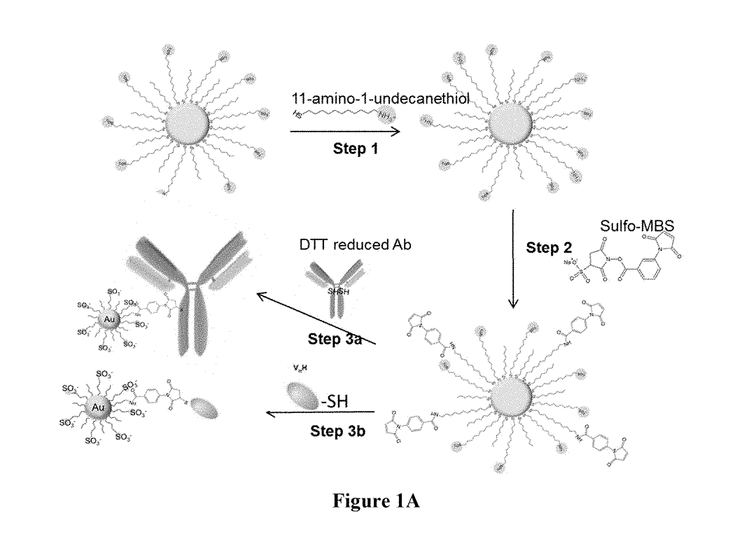

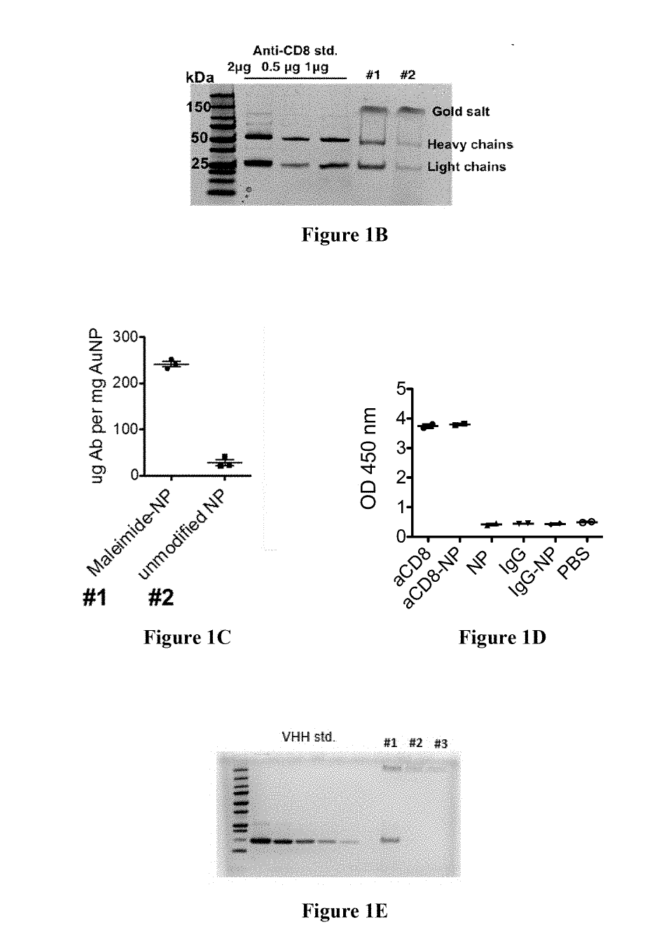

As illustrated in FIG. 1A, amphiphilic gold nanoparticles (amph-NPs) composed of MUS or mixed MUS and OT ligands (See, e.g., Irvine et al. Nano Letters 2013, 13, 4060-4067) were surface-modified with 11-Amino-1-undecanethiol hydrochloride. NMR quantification suggested 14% of total ligands on amph-NPs were replaced with amine ligands. Sulfo-MBS was mixed with amine-functionalized amph-NPs via N-hydroxysuccinimide (NHS)/amine chemistry. As a result, free maleimide groups were tagged onto nanoparticle surface. Finally, anti-CD8 antibodies mildly reduced with dithiothreitol (DTT) were coupled to maleimide-functionalized nanoparticles at a 1:2 or 1:1 molar ratio (see FIGS. 1B-1C). To test the functionality of antibodies post conjugation steps, ELISA against CD8 antigen was performed (FIG. 1D). Results showed that anti-CD8 on antibody-drug nanocarriers (ADNCs) remained fully functional in terms of affinity to CD8 antigens, whereas IgG coupled nanoparticles or free nanoparticles had low affinity to CD8 antigen.

### In Vitro and In Vivo Targeting Efficiency of Anti-CD8 Conjugates to CD8⁺ T Cells

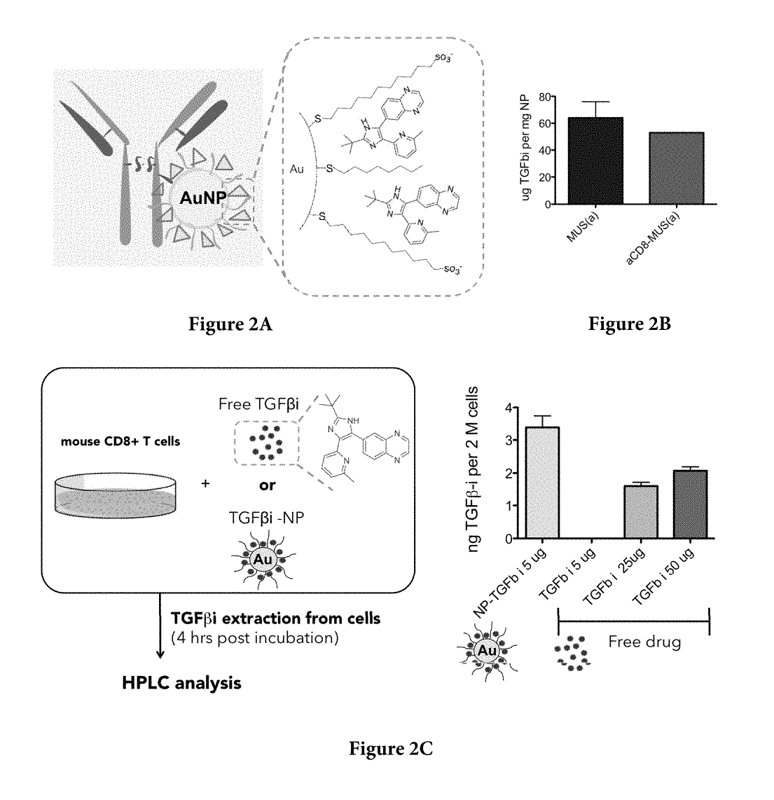

Many small molecules are under intensive investigation as new pharmaceuticals for cancer and infection treatments as well as other diseases and conditions. They are potent, have well-defined structures, and are often cost effective. However, many of them are not soluble in water and have intolerable off-target toxicity. If a hydrophobic molecule cannot traverse the milieu of aqueous environments and membranes en route to its cytosolic target, then the drug cannot be effective. While in vitro testing of various new hydrophobic small molecules can demonstrate desirable physiological effects, the same molecules generally suffer from exceptionally rapid clearance in vivo. Doses required to achieve the observed in vitro effects often cause systemic toxicity. Strategies to deliver concentrated small molecules via AuNPs to targeted sites would solve the issues associated with their soluble form. Here, it is demonstrated that small molecules are readily loaded onto amphiphilic nanoparticle ligand shells in ADNCs (FIGS. 2A-C).

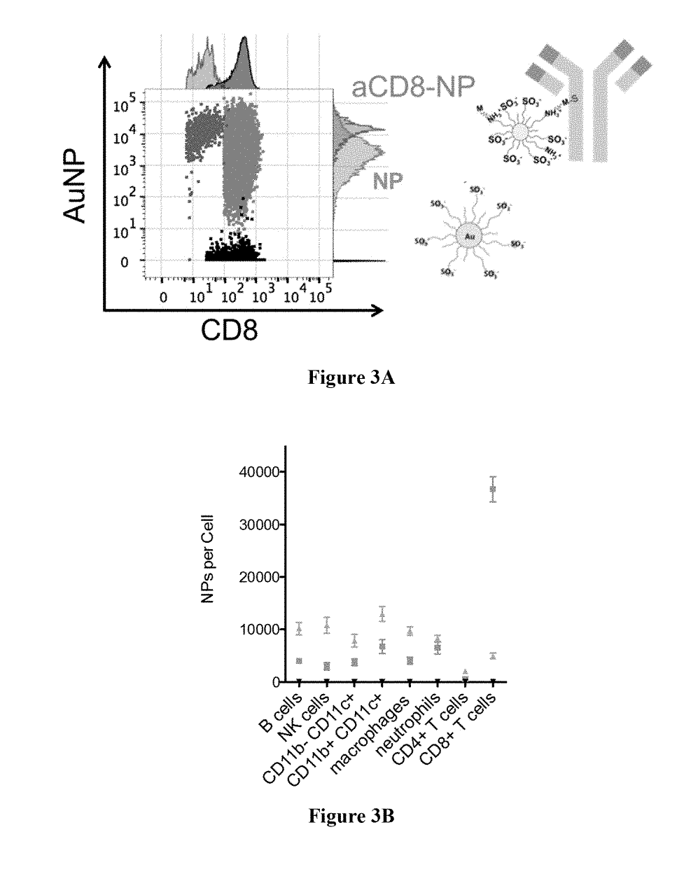

An initial experiment was established to test the targeting efficiency of anti-CD8 ADNCs to CD8⁺ T cells in vitro. Splenocytes were isolated and incubated with anti-CD8 ADNC or nanoparticles without antibody. As shown in FIGS. 3A-3B, anti-CD8 ADNCs (abbreviated “aCD8-NP”) increased nanoparticle delivery to the target cell by 9.2-fold, and decreased nanoparticle uptake in off-target cell types. Splenar CD8+ T cell marker intensity decreased in the group which cells were pretreated with anti-CD8 antibody conjugated nanoparticles, suggesting receptor-mediated nanoparticle delivery occurred.

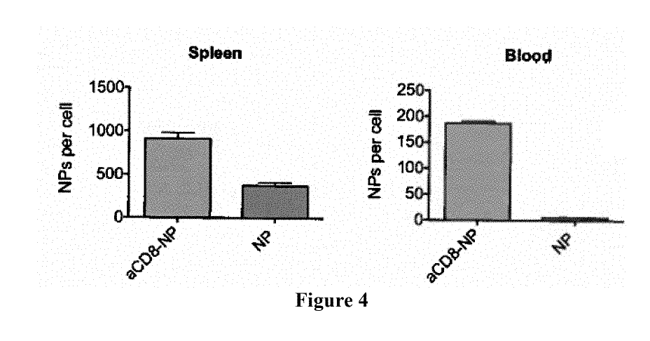

Next, in vivo targeting efficiency of anti-CD8 ADNCs was evaluated. Nanoparticles or anti-CD8 ADNCs (equivalent dose of free nanoparticles) were injected to C57BL/6 mice tail veins intravenously, and spleens and peripheral blood mononuclear cells (PBMCs) were isolated 24 hours post injection. ADNCs enhanced nanoparticle delivery to CD8⁺ T cells in the spleen by 2-fold; and in the blood by 35-fold (FIG. 4). While ADNCs slightly increased non-specific uptake by phagocytes (macrophages, dendritic cells and neutrophils) compared to bare nanoparticles, ADNCs resulted in dramatically reduced NP uptake in most non-phagocytic lymphocytes (B cells, CD4+ T cells, and NK cells). This resulted in an overall significantly improved on-target to off-target ratio. This embodiment of antibody conjugation for the purpose of targeting nanoparticles to a specific cell type is thus demonstrated and shown to exhibit significantly improved on-target absorption.

### Enhancement of Payload Quantity and Facilitation of Cytosolic Delivery

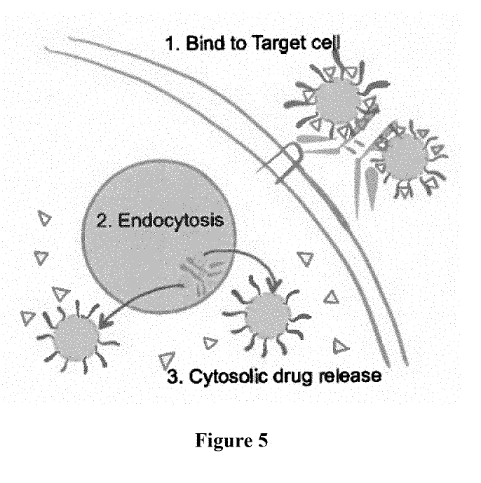

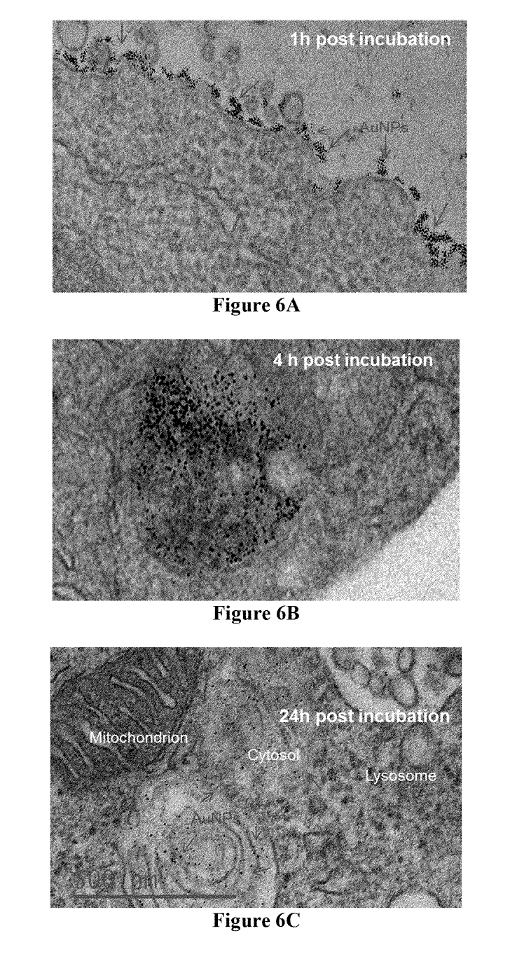

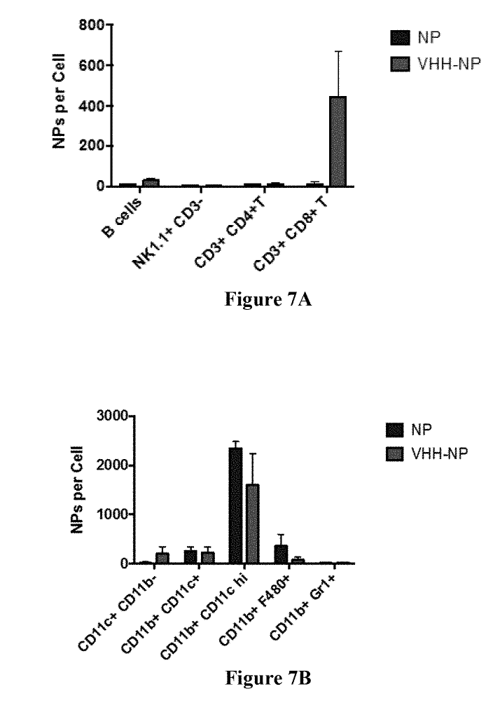

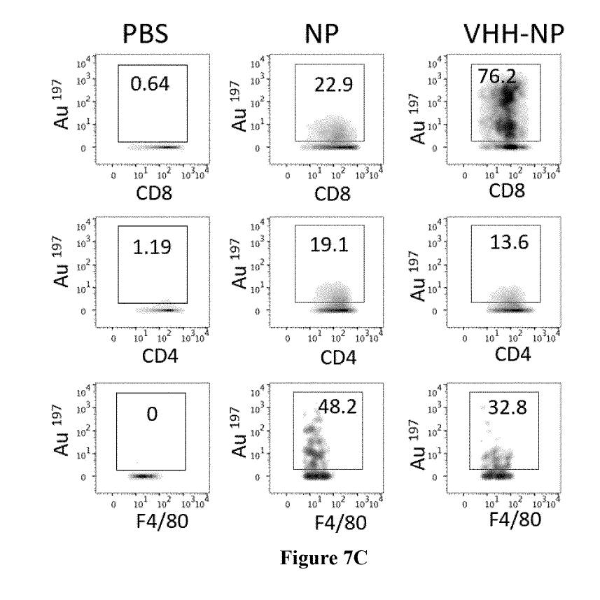

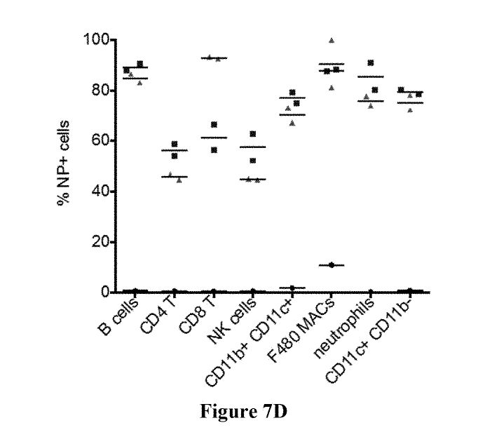

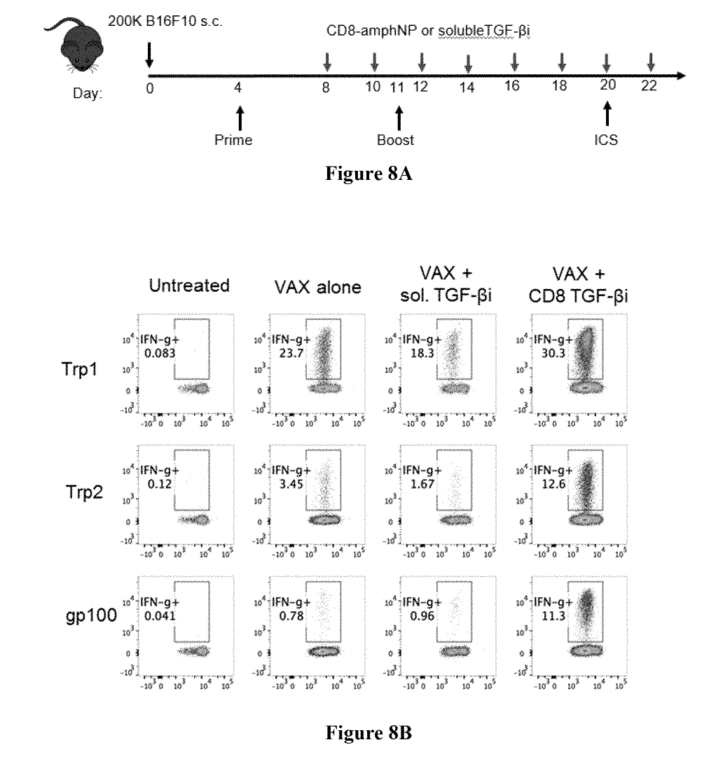

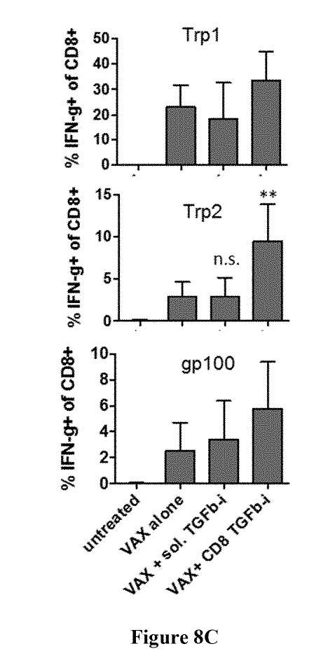

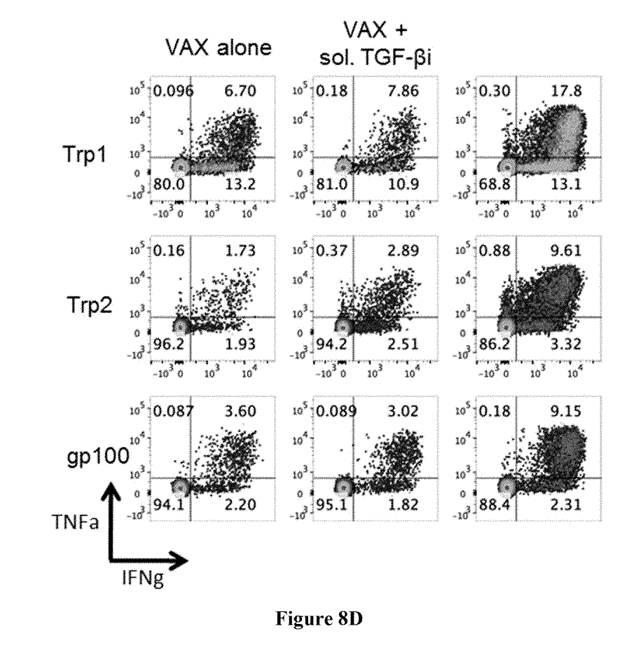

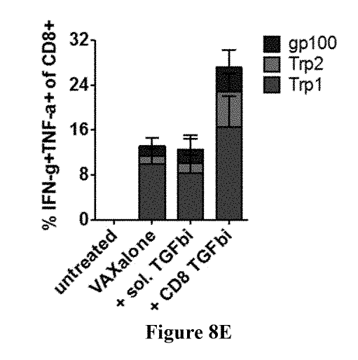

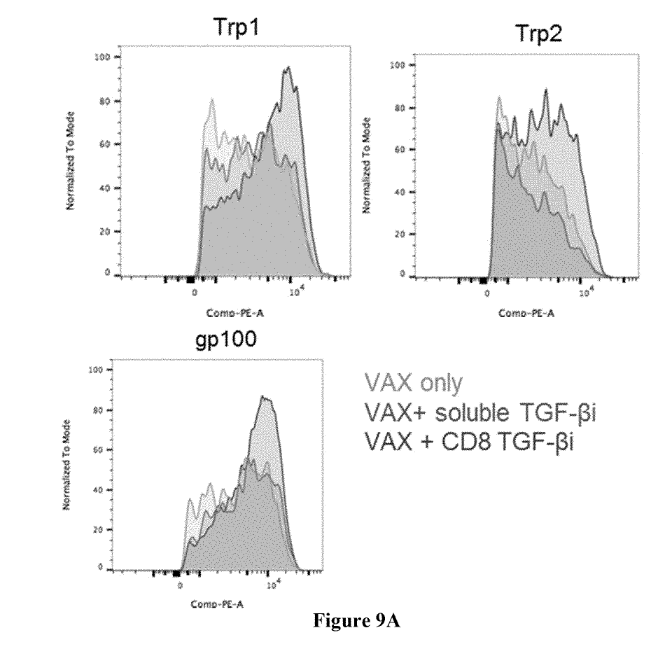

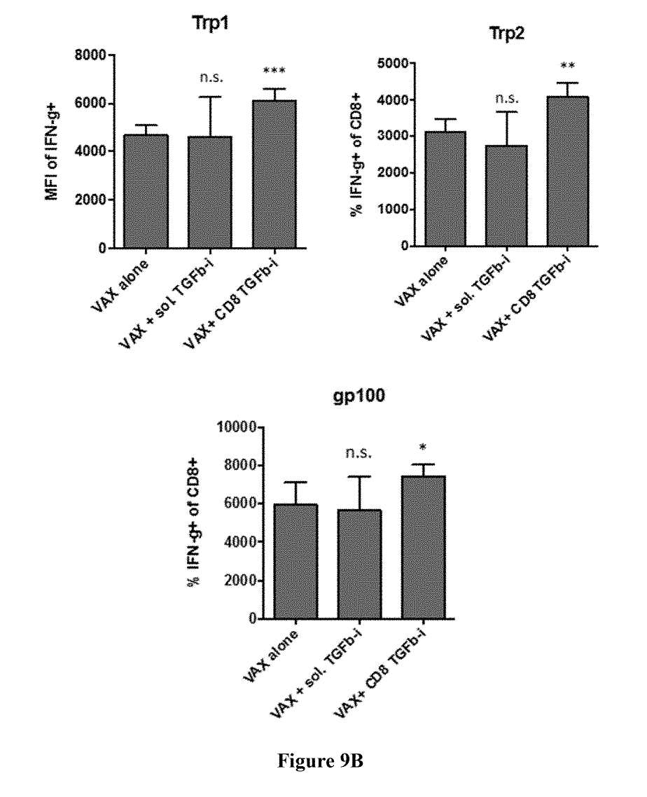

Nanoparticle-targeting agent conjugates of the present invention may provide a means to deliver small molecule immunomodulators or cytotoxins to enhance anti-cancer immunity, reverse autoimmune conditions, and combat infection. For example, ADNCs can load 50-200 molecules per nanoparticle (or per antibody), depending on the drug molecule's hydrophobicity and polarity. ADNCs enhanced nanoparticle uptake in CD8+ T cells in vitro and in vivo. The possible mechanisms of drug payload release by ADNCs is illustrated in FIG. 5:

(1) Drug-loaded ADNCs bind to target cell surface;

(2) Internalization of drug-loaded ADNCs via receptor mediated endocytosis. Amph-NPs get cleaved off from antibody due to reducing environment in endosomes that destabilize maleimide-sulfur bonds; and

(3) Drug loaded Amph-NPs embed in endosomal membranes and/or transit through intracellular membranes, resulting in ligand shell fluctuation which then allow drugs to be released into the cytosol.

Additionally, once the ADNC has reached the target cell membrane, the conjugated nanoparticle can utilize its membrane embedding properties to facilitate cytosolic delivery of hydrophobic drugs—something current antibody-drug conjugates (ADCs) have problems with due to the effect of hydrophobic molecules on their tertiary and quaternary structures reducing their stability and contributing to aggregation-induced clearance.

Critically, ADNCs offer significant advantages in comparison to current state-of-the-art antibody-drug conjugates (ADCs). For example, (1) greater diversity in deliverable payload type (many more molecule/drug candidates); and (2) an orders of magnitude increase in the quantity of molecules carried per antibody. Current ADCs can only deliver a very small number of payload molecules. This forces ADC treatments to use only the most potent classes of molecules, since a target cell cannot expect to receive very many. ADNCs can utilize drugs requiring higher treatment concentrations since ADNCs deliver orders of magnitude more payload molecules per antibody. This dramatically expands the pool of delivery candidates and allows many more ADNC applications than current ADCs. Thus, ADNCs may open up entire classes of molecules for drug discovery.

This invention has broad applications as a delivery system. ADNC targeting should generally be similar to the free antibody, and ADNCs can carry a wide variety of cargos. ADNC applications could be viable for: anti-cancer treatments, auto-immune disorder therapies, anti-pathogenic immunomodulation therapies, vaccines, and other applications.

### EQUIVALENTS AND SCOPE

Text here. In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process.

Furthermore, the invention encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should it be understood that, in general, where the invention, or aspects of the invention, is/are referred to as comprising particular elements and/or features, certain embodiments of the invention or aspects of the invention consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the invention, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment of the present invention that falls within the prior art may be explicitly excluded from any one or more of the claims. Because such embodiments are deemed to be known to one of ordinary skill in the art, they may be excluded even if the exclusion is not set forth explicitly herein. Any particular embodiment of the invention can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the present invention, as defined in the following claims.

1. A conjugate comprising one or more nanoparticles covalently linked to a targeting agent; wherein:
the nanoparticle comprises an organic outer shell;
one or more therapeutic agents are associated with the organic outer shell of the nanoparticle; and
the nanoparticle is less than approximately 10 nm in diameter.

2. The conjugate of claim 1, wherein the conjugate comprises one or two nanoparticles per targeting agent.

3-4. (canceled)

5. The conjugate of claim 1, wherein each nanoparticle is from 2-4 nm in diameter, inclusive.

6. (canceled)

7. The conjugate of claim 1, wherein the one or more nanoparticles are metal nanoparticles.

8. The conjugate of claim 1, wherein the one or more nanoparticles are transition metal nanoparticles.

9. The conjugate of claim 1, wherein the one or more nanoparticles are comprised of metals selected from the group consisting of gold, silver, copper, platinum, ruthenium, rhenium, and mixtures thereof.

10. (canceled)

11. The conjugate of claim 1, wherein the organic outer shell of each nanoparticle is an amphiphilic outer shell.

12-18. (canceled)

19. The conjugate of claim 1, wherein the organic outer shell of each nanoparticle comprises alkylsulfonate groups, unsubstituted alkyl groups, and alkylamino groups.

20. (canceled)

21. The conjugate of claim 1 comprising more than 50 molecules of the one or more therapeutic agents.

22. (canceled)

23. The conjugate of claim 1, wherein the one or more therapeutic agents are non-covalently adsorbed on the outer shells of the nanoparticles.

24. The conjugate of claim 1, wherein the one or more therapeutic agents are small molecules.

25-27. (canceled)

28. The conjugate of claim 1, wherein the targeting agent is linked to each nanoparticle with a crosslinking reagent.

29. (canceled)

30. The conjugate of claim 1, wherein each nanoparticle is smaller than the targeting agent.

31. The conjugate of claim 1, wherein the targeting agent is a peptide, protein, polymer, nucleic acid, small molecule, particle, or carbon nanostructure.

32. The conjugate of claim 1, wherein the targeting agent is an antibody or an antibody fragment.

33-36. (canceled)

37. The conjugate of claim 32, wherein the targeting agent is a single domain antibody.

38-40. (canceled)

41. A pharmaceutical composition comprising a conjugate of claim 1, and a pharmaceutically acceptable excipient.

42. A method of delivering one or more therapeutic agents to a cell, the method comprising contacting a cell with a conjugate of claim 1.

43-45. (canceled)

46. A method of treating a disease in a subject in need thereof, the method comprising administering to the subject a conjugate of claim 1.

47-53. (canceled)

54. A method of preparing a conjugate of claim 1, the method comprising the steps of:
(a) providing a nanoparticle comprising an organic outer shell, wherein the organic outer shell comprises one or more reactive moieties;
(b) contacting the nanoparticle provided in step (a) with a crosslinking reagent, thereby forming a covalent bond between the reactive moiety on the outer shell of the nanoparticle and the crosslinking reagent; and
(c) contacting the conjugate formed in step (b) with a targeting agent comprising one or more reactive moieties, thereby forming a covalent bond between the reactive moiety on the targeting agent and the crosslinking reagent.

55-66. (canceled)
